SEQUENCE LISTING (21 sequences)

SEQ ID NO: 1 - DNA, 738 bp, Streptococcus suis
atgacaacagtaaatgaagcattaaataatgtaagagctcaggttgggtccggtgtgtctgttggcaacggcgaatgctacgctttggctagttggtacgagcgcatgattagtccggatgcaactgtcggacttggcgctggtgtgggctgggtcagcggtgcaatcggcgatacaatctctgccaaaaacatcggctcatcatacaactggcaagctaacggctggacagtttccacatctggtccatttaaagcaggtcagattgtgacgcttggggcaacaccaggaaacccttacggacatgtggtaatcgtcgaagcagtggacggcgatagattgactattttggagcaaaactacggcgggaaacgttatcccgtccgtaattattacagcgctgcaagctatcgtcaacaggtcgtgcattacatcacaccgcctggcacggtcgcacagtcagcacccaaccttgcaggctctcgttcctatcgcgagacgggcactatgactgtcacggtcgatgctctcaatgttcgcagggggccaaatacttcaggcgagattgtagcagtatacaagcgtggtgaatcatttgactatgatactgtcatcatcgatgtcaatggctatgtctgggtgtcttacataggcggcagcggcaaacgtaactacgttgcgacgggcgctaccaaagacggtaagcgtttcggcaatgcttggggtacatttaaataa

SEQ ID NO: 2 - protein, 245 aa, Streptococcus suis
MTTVNEALNNVRAQVGSGVSVGNGECYALASWYERMISPDATVGLGAGVGWVSGAIGDTISAKNIGSSYNWQANGWTVSTSGPFKAGQIVTLGATPGNPYGHVVIVEAVDGDRLTILEQNYGGKRYPVRNYYSAASYRQQVVHYITPPGTVAQSAPNLAGSRSYRETGTMTVTVDALNVRRAPNTSGEIVAVYKRGESFDYDTVIIDVNGYVWVSYIGGSGKRNYVATGATKDGKRFGNAWGTFK

SEQ ID NO: 3 - protein, 245 aa, Artificial Sequence
  Description of Artificial Sequence Synthetic polypeptide
MTTVNEALNNVRAQVGSGVSVGNGECYALASWYERMISPDATVGLGAGVGWVSGAIGDTISAKNIGSSYNWQANGWTVSTSGPFKAGQIVTWGATPGNPYGHVSIVEAVDGDRLTILEQNYGGKRYPTRNYYSAASSRQQVVHYITPPGTVAQSAPNLAGSRSYRETGTMTVTVDALNVRRAPNTSGEIVAVYKRGESFDYDTVIIDVNGYVWVSYIGGSGKRNYVATGATKDGKRFGNAWGTFK

SEQ ID NO: 4 - protein, 245 aa, Artificial Sequence
  Description of Artificial Sequence Synthetic polypeptide
MTTVNEALNNVRAQVGSGVSVGNGECYALASWYERMISPDATVGLGAGVGWVSGAIGDTISAKNIGSSYNWQANGWTVSTSGPFKAGQIVTWGATPGNPYGHVSIVEAVDGDRLTILEQNYGGKRYPTRNYYSAASSRQQVVHYITPPGTVAQSAPNLAGSRSYRETGTMTVTVDALNVRRAPNTSGEIVAVYKRGEHFDYDTVIEDVNGYVWVSYIGGSGKRNYVATGATKDGKRFGNAWGTFK

SEQ ID NO: 5 - protein, 245 aa, Artificial Sequence
  Description of Artificial Sequence Synthetic polypeptide
MTTVNEALNNVRAQVGSGVSVGNGECYALASWYERMISPDATVGLGAGVGWVSGAIGDTISAKNIGSSYNWQANGWTVSTSGPFKAGQIVTWGATPGNPYGHVSIVEAVDGDRLTILEQNYGGKRYPTRNYYSAASSRQQVVHYITPPGTVAQSAPNLAGSRSYRETGTMTVTVDALNVRRAPNTSGEIVAVYKRGEQFDYDTAIIDVNGYAWVSYIGGSGKRNYVATGATKDGKRFGNAWGTFK

SEQ ID NO: 6 - protein, 245 aa, Artificial Sequence
  Description of Artificial Sequence Synthetic polypeptide
MTTVNEALNNVRAQVGSGVSVGNGECYALASWYERMISPDATVGLGAGVGWVSGAIGDTISAKNIGSSYNWQANGWTVSTSGPFKAGQIVTWGATPGNPYGHVSIVEAVDGDRLTILEQNYGGKRYPTRNYYSAASSRQQVVHYITPPGTVAQSAPNLAGSRSKRETGTMTVTVDALNVRRAPDTSGEIVAVYKRGEQFDYDTVIIDVNGYVWVSYIGGSGKRNYVATGATKDGKRFGNAWGTFK

SEQ ID NO: 7 - protein, 245 aa, Artificial Sequence
  Description of Artificial Sequence Synthetic polypeptide
MTTVNEALNNVRAQVGSGVSVGNGECYALASWYERMISPDATVGLGAGVGWVSGAIGDTISAKNIGSSYNWQANGWTVSTSGPFKAGQIVTWGATPGNPYGHVSIVEAVDGDRLTILEQNYGGKRYPTRNYYSAASSRQQVVHYITPPGTVAQSAPNLAGSRSKRETGTMTVTVDALNVRRAPDTSGEIVAVYKRGESFDYDTVIIDVNGYVWVSYIGGSGKRNYVATGATKDGKRFGNAWGTFK

SEQ ID NO: 8 - protein, 245 aa, Artificial Sequence
  Description of Artificial Sequence Synthetic polypeptide
MTTVNEALNNVRAQVGSGVSVGNGECYALASWYERMISPDATVGLGAGVGWVSGAIGDTISAKNIGSSYNWQANGWTVSTSGPFKAGQIVTWGATPGNPYGHVSIVEAVDGDRLTILEQNYGGKRYPTRNYYSAASSRQQVVHYITPPGTVAQSAPNLAGSRSNRETGTMTVTVDALNVRRAPNTSGEIVAVYKEGESFDYDTVIIDVNGYVWVSYIGGSGKRNYVATGATKDGKRFGNAWGTFK

SEQ ID NO: 9 - protein, 245 aa, Artificial Sequence
  Description of Artificial Sequence Synthetic polypeptide
MTTVNEALNNVRAQVGSGVSVGNGECYALASWYERMISPDATVGLGAGVGWVSGAIGDTISAKNIGSSYNWQANGWTVSTSGPFKAGQIVTWGATPGNPYGHVSIVEAVDGDRLTILEQNYGGKRYPTRNYYSAASSRQQVVHYITPPGTVAQSAPNLAGSRSYRETGTMTVTVDALNVRRAPDTSGEIVAVYKRGEHFDYDTVIIDVNGYVWVSYIGGSGKRNYVATGATKDGKRFGNAWGTFK

SEQ ID NO: 10 - protein, 245 aa, Artificial Sequence
  Description of Artificial Sequence Synthetic polypeptide
MTTVNEALNNVRAQVGSGVSVGNGECYALASWYERMISPDATVGLGAGVGWVSGAIGDTISAKNIGSSYNWQANGWTVSTSGPFKAGQIVTWGATPGNPYGHVSIVEAVDGDRLTILEQNYGGKRYPTRNYYSAASSRQQVVHYITPPGTVAQSAPNLAGSRSYRETGTMTVTVDALNVRRAPDTSGEIVAVYKRGESFDYDTAIIDVNGYAWVSYIGGSGKRNYVATGATKDGKRFGNAWGTFK

SEQ ID NO: 11 - protein, 245 aa, Artificial Sequence
  Description of Artificial Sequence Synthetic polypeptide
MTTVNEALNNVRAQVGSGVSVGNGECYALASWYERMISPDATVGLGAGVGWVSGAIGDTISAKNIGSSYNWQANGWTVSTSGPFKAGQIVTLGATPGNPYGHVVIVEAVDGDRLTILEQNYGGKRYPVRNYYSAASYRQQVVHYITPPGTVAQSAPNLAGSRSNRETGTMTVTVDALNVRRAPDTSGEIVAVYKEGESFDYDTEIIDVNGYVWVSYIGGSGKRNYVATGATKDGKRFGNAWGTFK

SEQ ID NO: 12 - protein, 245 aa, Artificial Sequence
  Description of Artificial Sequence Synthetic polypeptide
MTTVNEALNNVRAQVGSGVSVGNGECYALASWYEEMISPDATVGLGAGVGWVSGAIGDTISAKNIGSSYNWQANGWTVSTSGPFKAGQIVTWGATPGNPYGHVSIVEAVDGDRLTILEQNYGGKRYPTRNYYSAASSRQQVVHYITPPGTVAQSAPNLAGSRSYRETGTMTVTVDALNVRRAPNTSGEIVAVYKRGESFDYDTVIIDVNGYVWVSYIGGSGKRNYVATGATKDGKRFGNAWGTFK

SEQ ID NO: 13 - protein, 245 aa, Artificial Sequence
  Description of Artificial Sequence Synthetic polypeptide
MTTVNEALNNVRAQVGSGVSVGNGECYALASWYERMISPDATVGLGAGVGWVSGAIGDTISAKNIGSSYNWQANGWTVSTSGPFKAGQIVTLGATPGNPYGHVVIVEAVDGDRLTILEQNYGGKRYPTRNYYSAASSRQQVVHYITPPGTVAQSAPNLAGSRSKRETGTMTVTVDALNVRRAPNTSGEIVAVYKRGESFDYDTVIIDVNGYVWVSYIGGSGKRNYVATGATKDGKRFGNAWGTFK

SEQ ID NO: 14 - protein, 245 aa, Artificial Sequence
  Description of Artificial Sequence Synthetic polypeptide
MTTVNEALNNVRAQVGSGVSVGNGECYALASWYERMISPDATVGLGAGVGWVSGAIGDTISAKNIGSSYNWQANGWTVSTSGPFKAGQIVTWGATPGNPYGHVSIVEAVDGDRLTILEQNYGGKRYPTRNYYSAASSRQQVVHYITPPGTVAQSAPNLAGSRSKRETGTMTVTVDALNVRRAPNTSGEIVAVYKRGESFDYDTVIIDVNGYVWVSYIGGSGKRNYVATGATKDGKRFGNAWGTFK

SEQ ID NO: 15 - protein, 245 aa, Artificial Sequence
  Description of Artificial Sequence Synthetic polypeptide
MTTVNEALNNVRAQVGSGVSVGNGECYALASWYERMISPDATVGLGAGVGWVSGAIGDTISAKNIGSSYNWQANGWTVSTSGPFKAGQIVTWGATPGNPYGHVSIVEAVDGDRLTILEQNYGGKRYPTRNYYSAASSRQQVVHYITPPGTVAQSAPNLAGSRSKRETGTMTVTVDALNVRRAPNTSGEIVAVYKRGESFDYDTKIIDVNGYEWVSYIGGSGKRNYVATGATKDGKRFGNAWGTFK

SEQ ID NO: 16 - protein, 245 aa, Artificial Sequence
  Description of Artificial Sequence Synthetic polypeptide
MTTVNEALNNVRAQVGSGVSVGNGECYALASWYERMISPDATVGLGAGVGWVSGAIGDTISAKNIGSSYNWQANGWTVSTSGPFKAGQIVTWGATPGNPYGHVSIVEAVDGDRLTILEQNYGGKRYPTRNYYSAASSRQQVVHYITPPGTVAQSAPNLAGSRSKRETGTMTVTVDALNVRRAPDTSGEIVAVYKRGEQFDYDTKIIDVNGYEWVSYIGGSGKRNYVATGATKDGKRFGNAWGTFK

SEQ ID NO: 17 - protein, 245 aa, Artificial Sequence
  Description of Artificial Sequence Synthetic polypeptide
MTTVNEALNNVRAQVGSGVSVGNGECYALASWYERMISPDATVGLGAGVGWVSGAIGDTISAKNIGSSYNWQANGWTVSTSGPFKAGQIVTWGATPGNPYGHVSIVEAVDGDRLTILEQNYGGKRYPTRNYYSAASSRQQVVHYITPPGTVAQSAPNLAGSRSKRETGTMTVTVDALNVRRAPNTSGEIVAVYKRGESFDYDTVIEDVNGYVWGSYIGGSGKRNYVATGATKDGKRFGNAWGTFK

SEQ ID NO: 18 - protein, 244 aa, Streptococcus suis
TTVNEALNNVRAQVGSGVSVGNGECYALASWYERMISPDATVGLGAGVGWVSGAIGDTISAKNIGSSYNWQANGWTVSTSGPFKAGQIVTLGATPGNPYGHVVIVEAVDGDRLTILEQNYGGKRYPVRNYYSAASYRQQVVHYITPPGTVAQSAPNLAGSRSYRETGTMTVTVDALNVRRAPNTSGEIVAVYKRGESFDYDTVIIDVNGYVWVSYIGGSGKRNYVATGATKDGKRFGNAWGTFK

SEQ ID NO: 19 - protein, 139 aa, Streptococcus suis
LNNVRAQVGSGVSVGNGECYALASWYERMISPDATVGLGAGVGWVSGAIGDTISAKNIGSSYNWQANGWTVSTSGPFKAGQIVTLGATPGNPYGHVVIVEAVDGDRLTILEQNYGGKRYPVRNYYSAASYRQQVVHYIT

SEQ ID NO: 20 - protein, 67 aa, Streptococcus suis
RSYRETGTMTVTVDALNVRRAPNTSGEIVAVYKRGESFDYDTVIIDVNGYVWVSYIGGSGKRNYVAT

SEQ ID NO: 21 - protein, 150 aa, Streptococcus sp.
NLANAQAQVGKYIGDGQCYAWVGWWSARVCGYSISYSTGDPMLPLIGDGMNAHSIHLGWDWSIANTGIVNYPVGTVGRKEDLRVGAIWCATAFSGAPFYTGQYGHTGIIESWSDTTVTVLEQNILGSPVIRSTYDLNTFLSTLTGLITFK

(19) United States
(12) Patent Application Publication
(10) Pub. No.: US 2022/0160842 A1
(43) Pub. Date: May 26, 2022

(54) METHOD OF TREATING INFECTIVE ENDOCARDITIS

(71) Applicant: CONTRAFECT CORPORATION, Yonkers, NY (US)

(72) Inventor: Raymond SCHUCH, Mountain Lakes, NJ (US)

(21) Appl. No.: 17/440,916

(22) PCT Filed: Mar. 20, 2020
(86) PCT No.: PCT/US2020/024051

§ 371 Date: Sep. 20, 2021

Related U.S. Application Data
(60) Provisional application No. 62/965,720, filed on Jan. 24, 2020.
(60) Provisional application No. 62/898,379, filed on Sep. 10, 2019.
(60) Provisional application No. 62/849,093, filed on May 16, 2019.
(60) Provisional application No. 62/832,708, filed on Apr. 11, 2019.
(60) Provisional application No. 62/822,386, filed on Mar. 22, 2019.

(51) Int. Cl. A61K 38/47; A61K 38/14; A61K 38/12; A61K 31/431; A61K 31/5377; A61K 45/06

(52) U.S. Cl. CPC A61K 38/47; A61K 38/14; A61K 38/12; C12Y 302/01017; A61K 31/5377; A61K 45/06; A61K 31/431

(57) ABSTRACT

The present disclosure is directed to a method of treating or preventing infective endocarditis due to Gram-positive bacteria, such as S. aureus, which method includes administering a therapeutically effective amount of a combination of one or more antibiotics, optionally at a sub-Minimum Inhibitory Concentration (MIC) level, and a PlySs2 lysin, such as a single dose of PlySs2 lysin at a sub-MIC level, wherein the one or more antibiotics and the PlySs2 lysin are administered simultaneously or sequentially to a subject in need thereof in any order.

### CROSS-REFERENCE TO RELATED APPLICATIONS

This application claims the benefit of, and relies on the filing date of, U.S. provisional patent application No. 62/822,386, filed 22 Mar. 2019, U.S. provisional patent application No. 62/832,708, filed 11 Apr. 2019, U.S. provisional patent application No. 62/849,093, filed 16 May 2019, U.S. provisional patent application No. 62/898,379 filed 10 Sep. 2019, and U.S. provisional patent application No. 62/965,720 filed 24 Jan. 2020, the entire disclosures of each of which is incorporated herein by reference in its entirety.

### SEQUENCE LISTING

The instant application contains a Sequence Listing which has been submitted electronically in ASCII format and is hereby incorporated by reference in its entirety. Said ASCII copy, created on 20 Mar. 2020, is named 0341_0005-00-304_SL.txt and is 42,690 bytes in size.

### FIELD OF THE DISCLOSURE

The present disclosure relates generally to the treatment and prevention of infective endocarditis due to Gram-positive bacteria, including Staphylococcus aureus, such as methicillin-sensitive Staphylococcus aureus (MSSA) and methicillin-resistant Staphylococcus aureus (MRSA), using lysin(s) and one or more antibiotics in series.

### BACKGROUND

Infective endocarditis is an infection of the endocardium, the thin, smooth endothelial membrane that lines the inside of the chambers of the heart and forms the surface of the valves. This disease typically results from bacteria entering into the bloodstream and then settling in the heart. While the endothelial lining of healthy myocardium and heart valves are generally resistant to infection by bacteria, injured endothelial lining often is associated with the formation of platelet-fibrin thrombi, which serve as sites for bacteria to adhere and colonize, resulting in vegetative growths containing fibrin, platelets, leukocytes, red blood cell debris and high concentrations of bacteria.

Because the most common pathogens causing infective endocarditis are Gram-positive bacteria, such as Staphylococcus aureus, cell-wall inhibitors, such as β-lactam antibiotics and vancomycin, are often combined with e.g., synergistic doses of gentamicin to enhance the killing of bacteria. Most of the pathogens, however, produce biofilms containing the bacteria in an extracellular matrix that many antibiotics are not able to effectively penetrate. Consequently, elevated antibiotic plasma concentrations are typically needed over a prolonged period of time to achieve an effective antibiotic concentration. Unfortunately, side effects, particularly nephrotoxicity, can limit the use of antibiotics in the treatment of infective endocarditis. Moreover, even when intensive drug therapy is tolerated, eradicating the infection often remains difficult, requiring the need for surgery.

Given the high mortality rate associated with infective endocarditis (22-27% in six months), novel strategies are needed to treat this disease. These strategies should include drugs and/or biologics that are capable of disrupting biofilm architecture and/or reducing the need for high levels of antibiotics over long periods.

### SUMMARY

171 The present disclosure is directed to a method of treating or preventing infective endocarditis in a subject due to Gram-positive bacteria (e.g., Staphylococcus aureus, including methicillin-resistant S. aureus (MRSA)), which method includes: administering a therapeutically effective amount of a combination of one or more antibiotics and a PlySs2 lysin comprising SEQ ID NO: 2 or a variant thereof having at least 80% identity to SEQ ID NO: 2, wherein the one or more antibiotics and the PlySs2 lysin are administered in series to the subject in need thereof in any order.

### BRIEF DESCRIPTION OF THE DRAWINGS

FIG. 1 depicts the amino acid sequence of a lysin (SEQ ID NO:2) and a polynucleotide encoding the lysin (SEQ ID NO: 1) as described in the detailed description. SEQ ID NO:2 represents a 245 amino acid polypeptide, which is the predicted amino acid sequence based on the DNA sequence. The predicted amino acid sequence includes the initial methionine residue, which is removed during post-translational processing, leaving a 244-amino acid polypeptide.

FIG. 2 depicts the daptomycin dose response on bacterial burden in a heart valve, kidneys and spleen as described in the Examples.

FIG. 3 depicts the Methicillin-Resistant S. aureus (MRSA) densities in target tissues after treatment with a lysin of the disclosure at different times relative to daptomycin as described in the Examples.

FIG. 4 depicts different daptomycin and lysin dose administration strategies as described in the Examples.

FIGS. 5A-5D depict the bacterial burden in cardiac (heart valve) vegetations following different lysin dosing strategies in addition to daptomycin as described in the Examples. Dosing amounts are as follows: a CF-301 dose fraction of 0.7 mg/kg plus daptomycin (FIG. 5A), a CF-301 dose fraction of 0.35 mg/kg plus daptomycin (FIG. 5B), a CF-301 dose fraction of 0.09 mg/kg plus daptomycin (FIG. 5C) and a CF-301 dose fraction of0.06 mg/kg plus daptomycin (FIG. 5D).

FIG. 6 is an alignment between the CHAP domain of PlySs2 (SEQ ID NO: 2) and PlyC (SEQ ID NO: 21) as described in the detailed description.

### DETAILED DESCRIPTION

### Definitions

As used herein, the following terms and cognates thereof shall have the following meanings unless the context clearly indicates otherwise:

“Carrier” refers to a solvent, additive, excipient, dispersion medium, solubilizing agent, coating, preservative, isotonic and absorption delaying agent, surfactant, propellant, diluent, vehicle and the like with which an active compound is administered. Such carriers can be sterile liquids, such as water, saline solutions, aqueous dextrose solutions, aqueous glycerol solutions, and oils, including those of petroleum, animal, vegetable or synthetic origin, such as peanut oil, soybean oil, mineral oil, sesame oil and the like.

“Pharmaceutically acceptable carrier” refers to any and all solvents, additives, excipients, dispersion media, solubilizing agents, coatings, preservatives, isotonic and absorption delaying agents, surfactants, propellants, diluents, vehicles and the like that are physiologically compatible. The carrier(s) must be “acceptable” in the sense of not being deleterious to the subject to be treated in amounts typically used in medicaments. Pharmaceutically acceptable carriers are compatible with the other ingredients of the composition without rendering the composition unsuitable for its intended purpose. Furthermore, pharmaceutically acceptable carriers are suitable for use with subjects as provided herein without undue adverse side effects (such as toxicity, irritation, and allergic response). Side effects are “undue” when their risk outweighs the benefit provided by the composition. Non-limiting examples of pharmaceutically acceptable carriers or excipients include any of the standard pharmaceutical carriers such as phosphate buffered saline solutions, water, and emulsions such as oil/water emulsions and microemulsions. Suitable pharmaceutical carriers are described, for example, in “Remington's Pharmaceutical Sciences” by E. W. Martin, 18th Edition. The pharmaceutically acceptable carrier may be a carrier that does not exist in nature.

“Bactericidal” or “bactericidal activity” refers to the property of causing the death of bacteria or capable of killing bacteria to an extent of at least a 3-log 10 (99.9%) or a better reduction among an initial population of bacteria over an 18-24 hour period.

“Bacteriostatic” or “bacteriostatic activity” refers to the property of inhibiting bacterial growth, including inhibiting growing bacterial cells, thus causing a 2-log 10 (99%) or better and up to just under a 3-log reduction among an initial population of bacteria over an 18-24 hour period.

“Antibacterial” refers to both bacteriostatic and bactericidal agents.

“Antibiotic” refers to a compound having properties that have a negative effect on bacteria, such as lethality or reduction of growth. An antibiotic can have a negative effect on Gram-positive bacteria, Gram-negative bacteria, or both. By way of example, an antibiotic can affect cell wall peptidoglycan biosynthesis, cell membrane integrity or DNA or protein synthesis in bacteria.

“Drug resistant” refers generally to a bacterium that is resistant to the antibacterial activity of a drug. When used in certain ways, drug resistance may specifically refer to antibiotic resistance. In some cases, a bacterium that is generally susceptible to a particular antibiotic can develop resistance to the antibiotic, thereby becoming a drug resistant microbe or strain. A “multi-drug resistant” (“MDR”) pathogen is one that has developed resistance to at least two classes of antimicrobial drugs, each used as monotherapy. For example, certain strains of S. aureus have been found to be resistant to several antibiotics including methicillin and/or vancomycin (Antibiotic Resistant Threats in the United States, 2013, U.S. Department of Health and Services, Centers for Disease Control and Prevention). One skilled in the art can readily determine if a bacterium is drug resistant using routine laboratory techniques that determine the susceptibility or resistance of a bacterium to a drug or antibiotic.

“Effective amount” refers to an amount which, when applied or administered in an appropriate frequency or dosing regimen, is sufficient to prevent, reduce, inhibit or eliminate bacterial growth or bacterial burden or prevent, reduce or ameliorate the onset, severity, duration or progression of the disorder being treated (here Gram-positive bacterial pathogen growth or infection), prevent the advancement of the disorder being treated, cause the regression of the disorder being treated, or enhance or improve the prophylactic or therapeutic effect(s) of another therapy, such as antibiotic or bacteriostatic therapy.

“Co-administer” refers to the administration of two agents, such as a lysin, and an antibiotic or any other antibacterial agent in a sequential manner, as well as administration of these agents in a substantially simultaneous manner, such as in a single mixture/composition or in doses given separately, but nonetheless administered substantially simultaneously to the subject, for example at different times in the same day or 24-hour period. Such co-administration of two agents, such as a lysin with one or more additional antibacterial agents, can be provided as a continuous treatment lasting up to days, weeks, or months. Additionally, depending on the use, the co-administration need not be continuous or coextensive. For example, if the use were as a systemic antibacterial agent to treat, e.g., a bacterial ulcer or an infected diabetic ulcer, the lysin, could be administered only initially within 24 hours of an additional antibiotic use and then the additional antibiotic use may continue without further administration of the lysin.

“Subject” refers to a mammal, a plant, a lower animal, a single cell organism or a cell culture. For example, the term “subject” is intended to include organisms, e.g., prokaryotes and eukaryotes, which are susceptible to or afflicted with bacterial infections, for example Gram-positive bacterial infections. Examples of subjects include mammals, e.g., humans, dogs, cows, horses, pigs, sheep, goats, cats, mice, rabbits, rats, and transgenic non-human animals. In certain embodiments, the subject is a human, e.g., a human suffering from, at risk of suffering from, or susceptible to infection by Gram-positive bacteria, whether such infection be systemic, topical or otherwise concentrated or confined to a particular organ or tissue.

“Polypeptide” refers to a polymer made from amino acid residues and generally having at least about 30 amino acid residues. The term “polypeptide” is used herein interchangeably with the term “protein” and “peptide.” The term includes not only polypeptides in isolated form, but also active fragments and derivatives thereof. The term “polypeptide” also encompasses fusion proteins or fusion polypeptides comprising a lysin polypeptide, and maintaining, for example, a lysin function. Depending on context, a polypeptide or protein or peptide can be a naturally occurring polypeptide or a recombinant, engineered or synthetically produced polypeptide. A particular lysin polypeptide, for example, can be, e.g., derived or removed from a native protein by enzymatic or chemical cleavage, or can be prepared using conventional peptide synthesis techniques (e.g., solid phase synthesis) or molecular biology techniques (such as those disclosed in Sambrook, J. et al., Molecular Cloning: A Laboratory Manual, Cold Spring Harbor Press, Cold Spring Harbor, N.Y. (1989)) or can be strategically truncated or segmented yielding active fragments, maintaining e.g., lysin activity against the same or at least one common target bacterium.

“Fusion polypeptide” refers to an expression product resulting from the fusion of two or more nucleic acid segments, resulting in a fused expression product typically having two or more domains or segments, which typically have different properties or functionality. In a more particular sense, the term “fusion polypeptide” also refers to a polypeptide or peptide comprising two or more heterologous polypeptides or peptides covalently linked, either directly or via an amino acid or peptide linker. The polypeptides forming the fusion polypeptide are typically linked C-terminus to N-terminus, although they can also be linked C-terminus to C-terminus, N-terminus to N-terminus, or N-terminus to C-terminus. The term “fusion polypeptide” can be used interchangeably with the term “fusion protein. Thus, the open-ended expression “a polypeptide comprising” a certain structure includes larger molecules than the recited structure such as fusion polypeptides.

“Heterologous” refers to nucleotide or polypeptide sequences that are not naturally contiguous. For example, in the context of the present disclosure, the term “heterologous” can be used to describe a combination or fusion of two or more polypeptides wherein the fusion polypeptide is not normally found in nature, such as for example a lysin polypeptide and a cationic and/or a polycationic peptide, an amphipathic peptide, a sushi peptide (Ding et al. Cell Mol Life Sci., 65(7-8):1202-19 (2008)), a defensin peptide (Ganz, T. Nature Reviews Immunology 3, 710-720 (2003)), a hydrophobic peptide, and/or an antimicrobial peptide which may have enhanced lytic activity. Included in this definition are two or more lysin polypeptides or active fragments thereof. These can be used to make a fusion polypeptide with lytic activity.

“Active fragment” refers to a portion of a polypeptide that retains one or more functions or biological activities of the isolated polypeptide from which the fragment was taken, for example bactericidal activity against one or more Gram-positive bacteria, such as S. aureus.

“Synergistic” or “Superadditive” refers to a beneficial effect brought about by two substances in combination that exceeds the sum of the effects of the two agents working independently. In certain embodiments the synergistic or superadditive effect significantly, i.e., statistically significantly, exceeds the sum of the effects of the two agents working independently. One or both active ingredients may be employed at a sub-threshold level, i.e., a level at which if the active substance is employed individually produces no or a very limited effect. The effect can be measured by assays such as the checkerboard assay, described here.

“Treatment” refers to any process, action, application, therapy, or the like, wherein a subject, including a human being, is subjected to medical aid with the object of curing a disorder, eradicating a pathogen, or improving the subject's condition, directly or indirectly. Treatment also refers to reducing incidence, alleviating symptoms, eliminating recurrence, preventing recurrence, preventing incidence, reducing the risk of incidence, improving symptoms, improving prognosis or combinations thereof. “Treatment” may further encompass reducing the population, growth rate or virulence of the bacteria in the subject and thereby controlling or reducing a bacterial infection in a subject or bacterial contamination of an organ, tissue or environment. Thus, “treatment” that reduces incidence may, for example, be effective to inhibit growth of at least one Gram-positive bacterium in a particular milieu, whether it be a subject or an environment. On the other hand “treatment” of an already established infection refers to reducing the population, killing, inhibiting the growth, and/or eradicating, the Gram-positive bacteria responsible for an infection or contamination.

“Preventing” refers to the prevention of the incidence, recurrence, spread, onset or establishment of a disorder such as a bacterial infection. It is not intended that the present disclosure be limited to complete prevention or to prevention of establishment of an infection. In some embodiments, the onset is delayed, or the severity of a subsequently contracted disease or the chance of contracting the disease is reduced, and such constitutes examples of prevention.

“Contracted diseases” refers to diseases manifesting with clinical or subclinical symptoms, such as the detection of fever, sepsis or bacteremia, as well as diseases that may be detected by growth of a bacterial pathogen (e.g., in culture) when symptoms associated with such pathology are not yet manifest.

“Derivative,” in the context of a peptide or polypeptide or active fragment thereof, is intended to encompass, for example, a polypeptide modified to contain one or more-chemical moieties other than an amino acid that do not substantially adversely impact or destroy the polypeptide's activity, such as lysin activity. The chemical moiety can be linked covalently to the peptide, e.g., via an amino terminal amino acid residue, a carboxy terminal amino acid residue, or at an internal amino acid residue. Such modifications may be natural or non-natural. In certain embodiments, a non-natural modification may include the addition of a protective or capping group on a reactive moiety, addition of a detectable label, such as an antibody and/or fluorescent label, addition or modification of glycosylation, or addition of a bulking group such as PEG (pegylation) and other changes known to those skilled in the art. In certain embodiments, the non-natural modification may be a capping modification, such as N-terminal acetylations and C-terminal amidations. Exemplary protective groups that may be added to lysin polypeptides include, but are not limited to t-Boc and Fmoc. Commonly used fluorescent label proteins such as, but not limited to, green fluorescent protein (GFP), red fluorescent protein (RFP), cyan fluorescent protein (CFP), yellow fluorescent protein (YFP) and mCherry, are compact proteins that can be bound covalently or noncovalently to a polypeptide or fused to a polypeptide without interfering with normal functions of cellular proteins. In certain embodiments, a polynucleotide encoding a fluorescent protein is inserted upstream or downstream of the polynucleotide sequence. This will produce a fusion protein (e.g., Lysin Polypeptide::GFP) that does not interfere with cellular function or function of a polypeptide to which it is attached. Polyethylene glycol (PEG) conjugation to proteins has been used as a method for extending the circulating half-life of many pharmaceutical proteins. Thus, in the context of polypeptide derivatives, such as lysin polypeptide derivatives, the term “derivative” encompasses polypeptides, such as lysin polypeptides, chemically modified by covalent attachment of one or more PEG molecules. It is anticipated that lysin polypeptides, such as pegylated lysins, will exhibit prolonged circulation half-life compared to unpegylated polypeptides, while retaining biological and therapeutic activity.

“Percent amino acid sequence identity” refers to the percentage of amino acid residues in a candidate sequence that are identical with the amino acid residues in the reference polypeptide sequence, such as a lysin polypeptide sequence, after aligning the sequences and introducing gaps, if necessary, to achieve the maximum percent sequence identity, and not considering any conservative substitutions as a part of the sequence identity. Alignment for purposes of determining percent amino acid sequence identity can be achieved in various ways that are within the skill in the art, for example, using publicly available software such as BLAST or software available commercially for example from DNASTAR. Two or more polypeptide sequences can be anywhere from 0-100% identical, or any integer value there between. In the context of the present disclosure, two polypeptides are “substantially identical” when at least 80% of the amino acid residues (typically at least about 85%, at least about 90%, and typically at least about 95%, at least about 98%, or at least 99%) are identical. The term “percent (%) amino acid sequence identity” as described herein applies to peptides as well. Thus, the term “Substantially identical” will encompass mutated, truncated, fused, or otherwise sequence-modified variants of isolated polypeptides and peptides, such as those described herein, and active fragments thereof, as well as polypeptides with substantial sequence identity (e.g., at least 80%, at least 85%, at least 90%, at least 95% identity, at least 98% identity, or at least 99% identity as measured for example by one or more methods referenced above) as compared to the reference (wild type or other intact) polypeptide. Two amino acid sequences are “substantially homologous” when at least about 80% of the amino acid residues (typically at least about 85%, at least about 90%, at least about 95%, at least about 98% identity, or at least about 99% identity) are identical, or represent conservative substitutions. The sequences of polypeptides of the present disclosure, are substantially homologous when one or more, or several, or up to 10%, or up to 15%, or up to 20% of the amino acids of the polypeptide, such as the lysin polypeptides described herein, are substituted with a similar or conservative amino acid substitution, and wherein the resulting polypeptide, such as the lysins described herein, have at least one activity, antibacterial effects, and/or bacterial specificities of the reference polypeptide, such as the lysins described herein.

As used herein, a “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

“Biofilm” refers to bacteria that attach to surfaces and aggregate in a hydrated polymeric matrix that may be comprised of bacterial- and/or host-derived components. A biofilm is an aggregate of microorganisms in which cells adhere to each other on a biotic or abiotic surface. These adherent cells are frequently embedded within a matrix comprised of, but not limited to, extracellular polymeric substance (EPS). Biofilm EPS, which is also referred to as slime (although not everything described as slime is a biofilm) or plaque, is a polymeric conglomeration generally composed of extracellular DNA, proteins, and polysaccharides.

“Suitable” in the context of an antibiotic being suitable for use against certain bacteria refers to an antibiotic that was found to be effective against those bacteria even if resistance subsequently developed.

Infective Endocarditis

The present disclosure is directed to a method of treating or preventing infective endocarditis or infective endocarditis recurrence due to Gram-positive bacteria, such as Staphylococcus aureus, using conventional antibiotics and lysins, particularly sub-MIC quantities of lysins, as described herein.

In certain embodiments, the infective endocarditis of the present method is characterized by the presence of a biofilm. Such biofilms formed in vivo often exhibit a complex architecture, at least in part, due to their exposure to host defense mechanisms. Due to the difficulty in penetrating this architecture, many antibiotics and biologics are not effective in treating chronic diseases, such as infective endocarditis, that are associated with the presence of a biofilm. The present methods, however, may be efficaciously used to treat infective endocarditis, including those caused by biofilm-forming Gram-positive bacteria, as evidenced in the Examples.

Infective endocarditis as used herein refers to an infection of the endocardium, which is the inner lining of the heart chambers and heart valves. Infective endocarditis generally occurs when bacteria from another part of the body, such as the mouth, is spread through the bloodstream and attach to damaged areas in the heart, where it may form a biofilm.

Endocarditis may be diagnosed by any art known method. Typically, the modified Duke criteria are used (Table 1, from Cahill et al., Lancet, 2016, 387:882-893, which is herein incorporated by reference in its entirety). A diagnosis is indicated when two major, one major with three minor or five minor criteria are observed. Alternatively, if pathology specimens are available from a surgery, the diagnosis can be made using pathological criteria, i.e., histology or positive culture of vegetation or abscess tissue.

TABLE 1
Modified Duke Criteria for Diagnosis of Infective Endocarditis

Pathological criteria
Microorganisms on histology or culture of a vegetation or intracardiac abscess
Evidence of lesions; vegetation or intracardiac abscess showing active endocarditis on histology
Defined by the presence of a vegetation, abscess, or new partial dehiscence of prosthetic valve
New valvular regurgitation
Note-increase or change in pre-existing murmur is not sufficient

| Major clinical criteria | | Minor clinical criteria | |
|---|---|---|---|
| 1) | Blood cultures positive for infective | 1) | Predisposition: predisposing heart |
| | endocarditis | | condition, intravenous drug use |

| | | |
|---|---|---|
| Typical microorganisms consistent with | 2) | Fever: temperature >38° C. |
| infective endocarditis from two separate | 3) | Vascular phenomena; major arterial |
| blood cultures; | | emboli, septic pulmonary infarcts, |
| Staphylococcus aureus, viridans streptococci, | | mycotic aneurysm, intracranial |
| Streptococcus bovis, HACEK (hemophilus, | | hemorrhages, conjunctival |
| aggregatibacter, cardiobacterium, Eikenella | | hemorrhages, Janeway lesions |
| corrodens, kingella) group, or community | 4) | Immunological phenomena; |
| acquired enterococci, in the absence of a | | glomerulonephritis, Osler's nodes, |
| primary focus or | | Roth spots, rheumatoid factor |
| Microorganisms consistent with infective | 5) | Microbiological evidence: positive |
| endocarditis from persistently positive blood | | blood culture that does not meet a |
| cultures; | | major criterion or serological evidence |
| At least two positive blood cultures from | | of active infection with organism |
| blood samples drawn >12 h apart, or | | consistent with infective endocarditis |
| All of three, or most of ≥4 separate cultures | 6) | Diagnosis of infective endocarditis is |
| of blood (with first and last sample >1 h | | definite in the presence of one |
| apart) | | pathological criterion, or two major |
| or | | criteria, or one major and three minor |
| Single positive blood culture for Coxiella | | criteria, or five minor criteria |

| burnetii, or phase 1 IgG antibody titre >1:800 | | Diagnosis of infective endocarditis is possible |
|---|---|---|
| 2) | Evidence of endocardial involvement | in the presence of one major and one minor |
| 3) | Echocardiography positive for | criteria, or three minor criteria |
| | infective endocarditis | |

The present methods may be used to treat or prevent endocarditis due to the causative agents listed in Table 1, such as Staphylococcus aureus. The present methods may also be used to treat or prevent endocarditis due to the causative agents of infective endocarditis described in the Examples. Typical causative agents include members of the Staphylococcus genus such as coagulase-negative staphylococcal species (CoNS). As is known in the art, CoNS are gram-positive cocci that divide in irregular “grape-like” clusters and are differentiated from S. aureus by their inability to produce coagulase and coagulate rabbit plasma. CoNS species include Staphylococcus epidermidis, Staphylococcus lugdunensis, Staphylococcus haemolyticus, Staphylococcus capitis. Staphylococcus hominus and Staphylococcus warneri.

Additional typical Staphylococcus agents include Staphylococcus pseudintermedius, Staphylococcus sciuri, Staphylococcus simulans and Staphylococcus hyicus. Antibiotic-resistant bacteria including methicillin-resistant Staphylococcus aureus (MRSA), vancomycin resistant Staphylococcus aureus (VRSA), daptomycin-resistant Staphylococcus aureus (DRSA), and/or linezolid-resistant Staphylococcus aureus (LRSA) as well as altered antibiotic sensitivity bacteria comprising vancomycin intermediate-sensitivity Staphylococcus aureus (VISA) are also contemplated.

In addition, the present methods may be used to treat or prevent endocarditis due to the Streptococcus species as described in Table 1 and the examples, such as Streptococcus gordonii, Streptococcus mitis, Streptococcus oralis, Streptococcus intermedius, Streptococcus salivarius, Streptococcus pyogenes, Streptococcus agalactiae, Streptococcus dysgalactiae, Streptococcus pneunoniae, Streptococcus mutans, Streptococcus anginosus and Streptococcus sanguinis. Typical Streptococcus species include Streptococcus intermedius, Streptococcus pyogenes (Lancefield group A), Streptococcus agalactiae (Lancefield group B) and Streptococcus dysgalactiae (Lancefield group G).

The present method may be used to treat or prevent any type of infective endocarditis including prosthetic valve endocarditis, cardiac device infection and right-sided endocarditis. In some embodiments, the infective endocarditis is prosthetic valve endocarditis. Prosthetic valve endocarditis refers to an infection that typically occurs in 3-4% of patients within five years of prosthetic valve surgery and which affects mechanical and/or bioprosthetic valves. In some embodiments, prosthetic valve endocarditis is health-care acquired. Early prosthetic valve endocarditis (less than one year after initial surgery) predominantly occurs in the first 2 months after surgery and is most often due to coagulase-negative staphylococci or S. aureus. Beyond one year, the range of organisms causing prosthetic valve endocarditis is the same as in native valve endocarditis.

In some embodiments, the infective endocarditis is a cardiac device infection. Cardiac devices include permanent pacemakers, cardiac resynchronization therapy and implantable cardioverter defibrillators. The infection can involve the generator pocket, the device leads or the surrounding endocardial surface. Risk factors for cardiac device infection include haematoma formation at the incision site, renal failure, complex device implantation (compared with permanent pacemakers) and revision procedures in the absence of antibiotic prophylaxis. Signs of generator pocket infection include local cellulitis, discharge, dehiscence, or pain. Infection involving the leads or endocardium can cause fever, malaise, and sepsis.

In some embodiments, the infective endocarditis is right-sided endocarditis. Right-sided infective endocarditis is typically associated with intravenous drug users, subjects with cardiac device infection, subjects using central venous catheters, subjects with Human Immunodeficiency Virus (HIV), and subjects having congenital heart disease. In some embodiments, the tricuspid valve is affected in right-sided endocarditis. In addition to features of bacteremia including sepsis, patients often have respiratory symptoms resulting from pulmonary emboli, pneumonia, and pulmonary abscess formation. In some embodiments, patients with right-sided endocarditis, such as intravenous drug users, exhibit low compliance with standard treatments.

In some embodiments, the present methods are used to treat a subject at risk for acquiring infective endocarditis. Subjects at risk for acquiring infective endocarditis include those who have previously been diagnosed with infective endocarditis, subjects with a prosthetic heart valve, subjects with a cardiac device as defined herein, subjects older than 60 years of age, intravenous drug users and/or those with rheumatic heart disease.

Lysins

The present methods for treating and/or preventing infective endocarditis, including preventing a recurrence, comprise administering a lysin or active fragment thereof or a variant or derivative thereof as described herein to a subject in need thereof in combination with one or more antibiotics as also herein described. Lysins are bacteriophage-encoded hydrolytic enzymes that liberate progeny phage from infected bacteria by degrading peptidoglycan from inside the cell, causing lysis of the host bacterium. The present lysins may be used as antimicrobial agents to lyse pathogenic bacteria by attacking peptidoglycan from outside the bacterial cell. Typically, lysins are highly specific for bacterial species and rarely lyse non-target organisms, including commensal gut bacteria, which may be beneficial in maintaining gastrointestinal homeostasis.

In some embodiments, the present lysins or active fragments thereof or variants or derivatives thereof exhibit bacteriocidal and/or bacteriostatic activity against Gram-positive bacteria. In some embodiments, the present lysins or active fragments thereof or variants or derivatives thereof also exhibit a low propensity for resistance, suppress antibiotic resistance and/or exhibit synergy with conventional antibiotics. In other embodiments, the present lysins or active fragments thereof or variants or derivatives thereof inhibit bacterial agglutination, biofilm formation and/or reduce or eradicate biofilm, including biofilm in a subject with infective endocarditis.

The bacteriocidal activity of the present lysins or active fragments thereof or variants or derivatives thereof may be determined using any method known in the art. For example, the present lysins or active fragments thereof or variants or derivatives thereof may be assessed in vitro using time kill assays as described, for example, in Mueller, et al., 2004, Antimicrob Agents Chemotherapy, 48:369-377, which is herein incorporated by reference in its entirety.

The bacteriostatic activity of the present lysins or active fragments thereof or variants or derivatives thereof may also be assessed using any art-known method. For example, growth curves may be performed in e.g., cation adjusted Mueller Hinton II Broth supplemented in human serum (caMHB/50% HuS) to a final concentration of50% or in 100% serum. The Gram-positive bacteria may be suspended with lysin and culture turbidity can be measured at an optical density at 600 nm using, e.g. a SPECTRAMAX® M3 Multi-Mode Microplate reader (Molecular Devices) with e.g., readings every 1 minute for 11 hours at 24° C. with agitation. Doubling times can be calculated in the logarithmic-phase of cultures grown in flasks with aeration according to the method described in Saito et al, 2014, Antimicrob Agents Chemother 58:5024-5025, which is herein incorporated by reference in its entirety and compared to the doubling times of cultures in the absence of the present lysins or active fragments thereof or variants or derivatives thereof.

Inhibition of bacterial agglutination may be assessed using any method known in the art. For example, the method described in Walker et al. may be used, i.e., Walker et al., 2013, PLoS Pathog, 9:e1003819, which is herein incorporated by reference in its entirety.

Methods for assessing the ability of the lysins or active fragments thereof or variants or derivatives thereof to inhibit or reduce biofilm formation in vitro are well known in the art and include a variation of the broth microdilution minimum Inhibitory Concentration (MIC) method with modifications (See Ceri et al. 1999. J. Clin Microbial. 37:1771-1776, which is herein incorporated by reference in its entirety and Schuch et al., 2017, Antimicrob. Agents Chemother. 61, pages 1-18, which is herein incorporated by reference in its entirety.) In this method for assessing the Minimal Biofilm Eradicating Concentration (MBEC), fresh colonies of e.g., an S. aureus strain, are suspended in medium, e.g., phosphate buffer solution (PBS) diluted e.g., 1:100 in TSBg (tryptic soy broth supplemented with 0.2% glucose), added as e.g., 0.15 ml aliquots, to a Calgary Biofilm Device (96-well plate with a lid bearing 96 polycarbonate pegs; lnnovotech Inc.) and incubated e.g., 24 hours at 37° C. Biofilms are then washed and treated with e.g., a 2-fold dilution series of the lysin in e.g., TSBg at e.g., 37° C. for 24 hours. After treatment, wells are washed, air-dried at e.g., 37° C. and stained with e.g., 0.05% crystal violet for 10 minutes. After staining, the biofilms are destained in e.g., 33% acetic acid and the OD600 of e.g., extracted crystal violet is determined. The MBEC of each sample is the minimum lysin concentration required to remove >95% of the biofilm biomass assessed by crystal violet quantitation.

Suitable lysins for use with the present method include the PlySs2 lysins as described in WO 2013/170015, which is herein incorporated by reference in its entirety. As used herein, the terms “PlySs2 lysin”, “PlySs2 lysins”, “PlySs2” and “CF-301” are used interchangeably and encompass the PlySs2 lysin set forth herein as SEQ ID NO: 2 (with or without initial methionine residue) or an active fragment thereof or variants or derivatives thereof as described in WO 2013/170015. PlySs2, which was identified as an anti-staphylococcal lysin encoded within a prophage of the Streptococcus suis genome, exhibits bacteriocidal and bacteriostatic activity against the following exemplified bacteria.

TABLE 2
Reduction in Growth of Different Bacteria and
Relative kill with a lysin, PlySs2 (partial listing)*.

| Bacteria | Relative Kill with PlySs2 |
|---|---|
| Staphlyococcus aureus | +++ |
| (VRSA, VISA, MRSA, MSSA) | |
| Streptococcus suis | +++ |
| Staphlyococcus epidermis | ++ |
| Staphlyococcus simulans | +++ |
| Listeria monocytogenes | ++ |
| Enterococcus faecalis | ++ |
| Streptococcus dysgalactiae | ++ |
| Streptococcus agalactiae | +++ |
| Streptococcus pyogenes | +++ |
| Streptococcus equi | ++ |
| Streptococcus sangunis | ++ |
| Streptococcus gordonii | ++ |
| Streptococcus sobrinus | + |
| Streptococcus rattus | + |
| Streptococcus oralis | + |
| Streptococcus pneumoniae | + |
| Bacillus thuringiensis | − |
| Bacillus cereus | − |
| Bacillus subtilis | − |
| Bacillus anthracis | − |
| Escherichia coli | − |
| Enterococcus faecium | − |
| Pseudomonas aeruginosa | − |

*Additional species are described in Example 1.

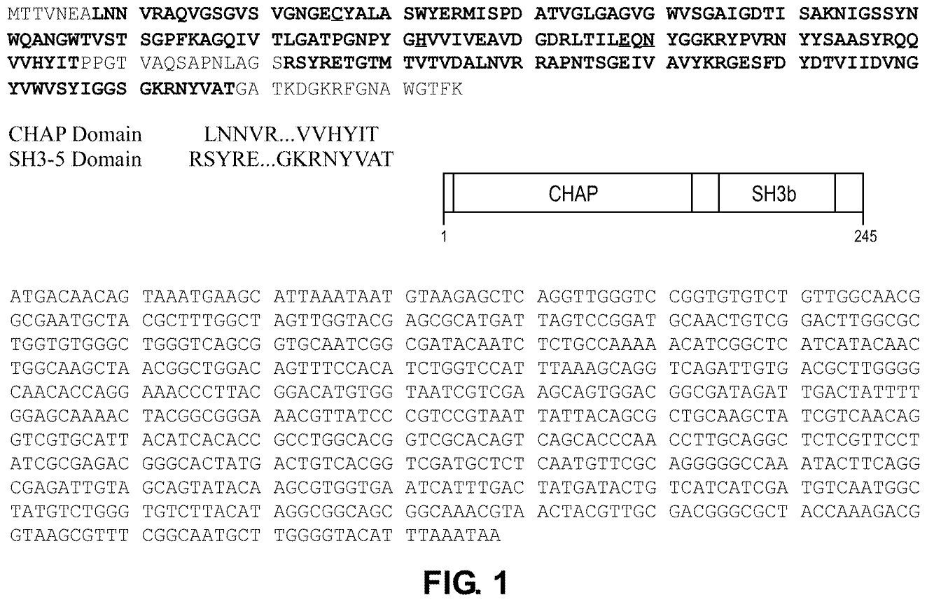

A particularly typical lysin for use with the present method is the PlySs2 lysin of SEQ ID NO: 2, or, more typically, the mature form of the PlySs2, which does not include the initial methionine residue, as set forth in SEQ ID NO: 18. The PlySs2 lysin of SEQ ID NOS: 2 and 18 has a domain arrangement characteristic of most bacteriophage lysins, defined by a catalytic N-terminal domain (SEQ ID NO: 19) linked to a cell wall-binding C-terminal domain (SEQ ID NO: 20). The N-terminal domain belongs to the cysteine-histidine-dependent amidohydrolases/peptidases (CHAP) family common among lysins and other bacterial cell wall-modifying enzymes. The C-terminal domain belongs to the SH3b family that typically forms the cell wall-binding element of lysins. FIG. 1 depicts the PlySs2 lysin of SEQ ID NO: 2 with the N- and C-terminal domains shown as bolded regions. The N-terminal CHAP domain corresponds to the first bolded amino acid sequence region starting with LNN and the C-terminal SH3b domain corresponds to the second bolded region starting with RSY.

In some embodiments, the present method comprises the administration of a variant lysin to a subject in need thereof. Suitable lysin variants for use with the present method include those polypeptides having at least one substitution, insertion and/or deletion in reference to SEQ ID NO: 2 or SEQ ID NO: 18 that retain at least one biological function of the reference lysin. In some embodiments, the variant lysins exhibit antibacterial activity including a bacteriolytic and/or bacteriostatic effect against a broad range of Gram-positive bacteria, including S. aureus and an ability to inhibit agglutination, inhibit biofilm formation and/or reduce biofilm. In some embodiments, the present lysin variants render Gram-positive bacteria more susceptible to antibiotics.

In some embodiments, a lysin variant suitable for use with the present methods includes an isolated polypeptide sequence having at least 80%, such as at least 85%, such as at least 90%, such as at least 95%, such as at least 98% or such as at least 99% sequence identity with SEQ ID NO: 2 or SEQ ID NO: 18, wherein the variant lysin retains one or more biological activities, e.g., catalytic activity, ability to bind to bacterial cell walls, such as Staphylococcus or Streptococcus, bacteriocidal or bacteriostatic activity, including the ability to kill Gram-positive bacteria in biofilm, such as Staphylococcus and/or Streptococcus of the PlySs2 lysin having the amino acid sequence of SEQ ID NO: 2 or SEQ ID NO: 18 as described herein.

Lysin variants may be formed by any method known in the art and as described in WO 2013/170015, which is herein incorporated by reference in its entirety, e.g., by modifying the PlySs2 lysin of SEQ ID NO: 2 or SEQ ID NO: 18 through site-directed mutagenesis or via mutations in hosts that produce the PlySs2 lysin of SEQ ID NO: 2 or SEQ ID NO: 18, and which retain one or more of the biological functions as described herein. For example, one of skill in the art can reasonably make and test substitutions or replacements to, e.g., the CHAP domain and/or the SH3b domain of the PlySs2 lysin of SEQ ID NO: 2 or SEQ ID NO: 18. Sequence comparisons to the Genbank database can be made with either or both of the CHAP and/or SH3b domain sequences or with the PlySs2 lysin full amino acid sequence of SEQ ID NO: 2 or SEQ ID NO: 18, for instance, to identify amino acids for substitution. For example, a mutant or variant having an alanine replaced for valine at valine amino acid residue 19 in the PlySs2 amino acid sequence of SEQ ID NO: 2 or SEQ ID NO: 18 is active and capable of killing Gram-positive bacteria in a manner similar to and as effective as the SEQ ID NO: 2 PlySs2 lysin.

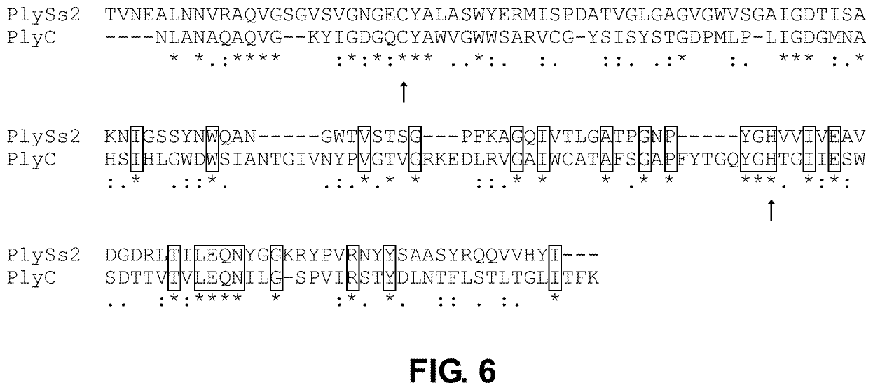

Further, as indicated in FIG. 1, the CHAP domain contains conserved cysteine and histidine amino acid sequences (the first cysteine and histidine in the CHAP domain) which are characteristic and conserved in CHAP domains of different polypeptides. It is reasonable to predict, for example, that the conserved cysteine and histidine residues should be maintained in a mutant or variant of PlySs2 so as to maintain activity or capability. Accordingly, particularly desirable residues to retain in a lysin variant of the present disclosure include active-site residues Cys26, His102, Glu118, and Asn120 in the CHAP domain of SEQ ID NO: 2. Particularly desirable substitutions include: Lys for Arg and vice versa such that a positive charge may be maintained, Glu for Asp and vice versa such that a negative charge may be maintained, Ser for Thr such that a free —OH can be maintained and Gln for Asn such that a free NH2 can be maintained. Other suitable variants include substitutions in SEQ ID NO: 2 or SEQ ID NO: 18 in the CHAP and/or SH3 domain regions that are not shared between other known lysins, such as between the CHAP domain of instant SEQ ID NO: 2 and the CHAP domain of PlyC as shown in for example, in Schmitz, 2011, “Expanding the Horizons of Enzybiotic Identification” Student Theses and Dissertations, paper 138, which is herein incorporated by reference in its entirety and depicted herein in FIG. 6.

Suitable variant lysins are also described in PCT Published Application No. WO 2019/165454 (International Application No.: PCT/US2019/019638), which is herein incorporated by reference in its entirety. Particularly, suitable variant lysins include those set forth herein as SEQ ID NOS: 3-17 as well as variant lysins having at least 80%, such as at least 85%, such as at least 90%, such as at least 95%, such as at least 98% or such as at least 99% sequence identity with any one of SEQ ID NOS: 3-17, wherein the variant lysin retains one or more biological activities of the PlySs2 lysin having the amino acid sequence of SEQ ID NO: 2 as described herein.

SEQ ID NOs: 3-17 are modified lysin polypeptides having at least one amino acid substitution relative to a counterpart wild-type PlySs2 lysin (SEQ ID NO: 2), while preserving antibacterial activity and effectiveness. SEQ ID NOs: 3-17 may be described by reference to their amino acid substitutions with respect to SEQ ID NO: 2, as shown below in Table A. The amino acid sequences of the modified lysin polypeptides (referencing differences from SEQ ID NO: 2 and the positions of its amino acid residues) are summarized using one-letter amino acid codes as follows:

TABLE A

| | Substitution location | | | | | | | |
|---|---|---|---|---|---|---|---|---|
| pp55 | | L92W | V104S | V128T | | | | |
| (SEQ ID NO: 3) | | | | and | | | | |
| | | | | Y137S | | | | |
| pp61 | | L92W | V104S | V128T | | | S198H | I206E |
| (SEQ ID NO: 4) | | | | and | | | | |
| | | | | Y137S | | | | |
| pp65 | | L92W | V104S | V128T | | | S198Q | V204A |
| (SEQ ID NO: 5) | | | | and | | | | and |
| | | | | Y137S | | | | V212A |
| pp296 | | L92W | V104S | V128T | Y164K | N184D | S198Q | |
| (SEQ ID NO: 6) | | | | and | | | | |
| | | | | Y137S | | | | |
| pp324 | | L92W | V104S | V128T | Y164N | N184D | | |
| (SEQ ID NO: 7) | | | | and | | | | |
| | | | | Y137S | | | | |
| pp325 | | L92W | V104S | V128T | Y164N | | R195E | |
| (SEQ ID NO: 8) | | | | and | | | | |
| | | | | Y137S | | | | |
| pp338 | | L92W | V104S | V128T | | N184D | S198H | |
| (SEQ ID NO: 9) | | | | and | | | | |
| | | | | Y137S | | | | |
| pp341 | | L92W | V104S | V128T | | N184D | | V204A |
| (SEQ ID NO: 10) | | | | and | | | | and |
| | | | | Y137S | | | | V212A |
| pp388 | | | | | Y164N | N184D | R195E | V204K |
| (SEQ ID NO: 11) | | | | | | | | and |
| | | | | | | | | V212E |
| pp400 | R35E | L92W | V104S | V128T | | | | |
| (SEQ ID NO: 12) | | | | and | | | | |
| | | | | Y137S | | | | |
| pp616 | | | | V128T | Y164K | | | |
| (SEQ ID NO: 13) | | | | and | | | | |
| | | | | Y137S | | | | |
| pp619 | | L92W | V104S | V128T | Y164K | | | |
| (SEQ. ID NO: 14) | | | | and | | | | |
| | | | | Y137S | | | | |
| pp628 | | L92W | V104S | V128T | Y164K | | | V204K |
| (SEQ. ID NO: 15) | | | | and | | | | and |
| | | | | Y137S | | | | V212E |
| pp632 | | L92W | V104S | V128T | Y164K | N184D | S198Q | V204K |
| (SEQ. ID NO: 16) | | | | and | | | | and |
| | | | | Y137S | | | | V212E |
| pp642 | | L92W | V104S | V128T | Y164K | | | I206E |
| (SEQ. ID NO: 17) | | | | and | | | | and |
| | | | | Y137S | | | | V214G |

In some embodiments the present method includes administering an active fragment of a lysin to a subject in need thereof. Suitable active fragments include those that retain a biologically active portion of a protein or peptide fragment of the embodiments, as described herein. Such variants include polypeptides comprising amino acid sequences that include fewer amino acids than the full length protein of the lysin protein and exhibit at least one activity of the corresponding full-length protein. Typically, biologically active portions comprise a domain or motif with at least one activity of the corresponding protein. An exemplary domain sequence for the N-terminal CHAP domain of the PlySs2 lysin is provided in FIG. 1 and SEQ ID NO: 19. An exemplary domain sequence for the C terminal SH3b domain of the PlySs2 lysin is provided in FIG. 1 and SEQ ID NO: 20. A biologically active portion of a protein or protein fragment of the disclosure can be a polypeptide which is, for example, 10, 25, 50, 100 amino acids in length. Other biologically active portions, in which other regions of the protein are deleted can be prepared by recombinant techniques and evaluated for one or more of the functional activities of the native form of a polypeptide of the embodiments.

In some embodiments, suitable active fragments include those having at least 80%, such as at least 85%, such as at least 90%, such as at least 95%, such as at least 98% or such as at least 99% sequence identity with the active fragments described herein including SEQ ID NO: 19 or 20, wherein the active fragment thereof retains at least one activity of CHAP and/or the SH3b domain.

A lysin or active fragment thereof or variant or derivative thereof as described herein for use in the present method may be produced by a bacterial organism after being infected with a particular bacteriophage or may be produced or prepared recombinantly or synthetically, e.g., chemically synthesized or prepared using a cell free synthesis system. In as much as the lysin polypeptide sequences and nucleic acids encoding the lysin polypeptides are described and referenced herein, the present lysins may be produced via the isolated gene for the lysin from the phage genome, putting the gene into a transfer vector, and cloning said transfer vector into an expression system, using standard methods of the art, as described for example in WO 2013/170015, which is herein incorporated by reference in its entirety. The present lysin variants may be truncated, chimeric, shuffled or “natural,” and may be in combination as described, for example, in U.S. Pat. No. 5,604,109, which is incorporated herein in its entirety by reference.

Mutations can be made in the amino acid sequences, or in the nucleic acid sequences encoding the polypeptides and lysins described herein, including in the lysin sequence set forth in SEQ ID NO: 2, SEQ ID NO: 18 or in active fragments or truncations thereof, such that a particular codon is changed to a codon which codes for a different amino acid to obtain a sequence with a substituted amino acid, or one or more amino acids are deleted or added.

Such a mutation is generally made by making the fewest nucleotide changes possible. A substitution mutation of this sort can be made to change an amino acid in the resulting protein in a non-conservative manner (for example, by changing the codon from an amino acid belonging to a grouping of amino acids having a particular size or characteristic to an amino acid belonging to another grouping) or in a conservative manner (for example, by changing the codon from an amino acid belonging to a grouping of amino acids having a particular size or characteristic to an amino acid belonging to the same grouping). Such a conservative change generally leads to less change in the structure and function of the resulting protein. A non-conservative change is more likely to alter the structure, activity or function of the resulting protein. The present disclosure should be considered to include sequences containing conservative changes which do not significantly alter the activity or binding characteristics of the resulting protein. Thus, one of skill in the art, based on a review of the sequence of the PlySs2 lysin polypeptide provided herein and on their knowledge and the public information available for other lysin polypeptides, can make amino acid changes or substitutions in the lysin polypeptide sequence. Amino acid changes can be made to replace or substitute one or more, one or a few, one or several, one to five, one to ten, or such other number of amino acids in the sequence of the lysin(s) provided herein to generate mutants or variants thereof. Such mutants or variants thereof may be predicted for function or tested for function or capability for anti-bacterial activity as described herein against, e.g., Staphylococcal, Streptococcal, or Enterococcal bacteria, and/or for having comparable activity to the lysin(s) as described and particularly provided herein. Thus, changes made to the sequence of lysin, and mutants or variants described herein can be tested using the assays and methods known in the art and described herein. One of skill in the art, on the basis of the domain structure of the lysin(s) hereof can predict one or more, one or several amino acids suitable for substitution or replacement and/or one or more amino acids which are not suitable for substitution or replacement, including reasonable conservative or non-conservative substitutions.

Antibiotics

The methods of treating or preventing infective endocarditis described herein comprise co-administering a therapeutically effective amount of one or more antibiotics and a PlySs2 lysin. In some embodiments, co-administration of a lysin or active fragment thereof or variant or derivative thereof and one or more antibiotic as described herein results in a synergistic bacteriocidal and/or bacteriostatic effect on Gram-positive bacteria such as S. aureus. Typically, the co-administration results in a synergistic effect on bacteriostatic and/or bactericidal activity. In other embodiments, the co-administration is used to suppress virulence phenotypes including biofilm formation and/or agglutination. In some embodiments, the co-administration is used to reduce an amount of biofilm in a subject.

Suitable antibiotics for use with the present methods include antibiotics of different types and classes, such as beta-lactams including penicillins (e.g. methicillin, oxacillin), cephalosporins (e.g. cefalexin and cefactor), monobactams (e.g. aztreonam) and carbapenems (e.g. imipenem and entapenem); macrolides (e.g. erythromycin, azithromycin), aminoglycosides (e.g. gentamicin, tobramycin, amikacin), glycopeptides (e.g., vancomycin, teicoplanin), oxazolidinones (e.g linezolid and tedizolid), lipopeptides (e.g. daptomycin) and sulfonamides (e.g. sulfamethoxazole).

Typically, vancomycin, daptomycin, linezolid and oxacillin are used with the present methods. Even more typically, daptomycin is used.

Dosages and Administration

Dosages of the present lysins or active fragments thereof or variants or derivatives thereof that are administered to a subject in need thereof depend on a number of factors including the activity of infection being treated, the age, health and general physical condition of the subject to be treated, the activity of a particular lysin or active fragment thereof or variant or derivative thereof, the nature and activity of the antibiotic, if any, with which a lysin or active fragment thereof or variant or derivative thereof according to the present disclosure is being paired and the combined effect of such pairing. Generally, effective amounts of the present lysins or active fragments thereof or variants or derivatives thereof to be administered are anticipated to fall within the range of 0.1-50 mg/kg (or 1 to 50 mcg/ml). The present lysins or active fragments thereof or variants or derivatives thereof may be administered according to any desired frequency or duration. For example, the present lysins or active fragments thereof or variants or derivatives thereof may be administered 1-4 times daily for a period up to 14 days. Typically, only a single dosage is administered. The antibiotic may be administered at standard dosing regimens or in lower amounts in view of the synergy. All such dosages and regimens however (whether of the lysin or active fragment thereof or variant or derivative thereof or any antibiotic administered in conjunction therewith) are subject to optimization. Optimal dosages can be determined by performing in vitro and in vivo pilot efficacy experiments as is within the skill of the art but taking the present disclosure into account.

Typically, the dosage of the lysin or active fragment thereof or variant or derivative thereof ranges from about 0.000025 to about 1.8 mg/kg, such as about 0.0.05 mg/kg to about 0.5 mg/kg or about 0.1 mg/kg to about 0.3 mg/kg. More typically, in healthy individuals, the dosage range is about 0.2 mg/kg to about 0.3 mg/kg, such as 0.25 mg/kg. In some embodiments, for example, in individuals with moderate and severe renal impairment, the dosage may be lower, e.g. 0.1 mg/kg to 0.2 mg/kg, such as 0.12 mg/kg. In some embodiments, the dosages, such as a single dosage, are administered intravenously over, for example, a two hour period.

It is contemplated that the present lysins or active fragments thereof or variants or derivatives thereof provide a bactericidal and, when used in smaller amounts, a bacteriostatic effect, and are active against a range of antibiotic-resistant bacteria and are not associated with evolving resistance. Based on the present disclosure, in a clinical setting, the present lysins or active fragments thereof or variants or derivatives thereof are a potent alternative (or additive or component) of compositions for treating endocarditis infections arising from drug- and multidrug-resistant bacteria when combined with certain antibiotics (even antibiotics to which resistance has developed). Existing resistance mechanisms for Gram-positive bacteria should not affect sensitivity to the lytic activity of the present polypeptides.

For any polypeptide of the present disclosure, the therapeutically effective dose can be estimated initially either in cell culture assays or in animal models, usually mice, rabbits, dogs, or pigs. The animal model can also be used to achieve a desirable concentration range and route of administration. Obtained information can then be used to determine the effective doses, as well as routes of administration in humans. However, typically systemic administration, in particular intravenous administration, is used. Dosage and administration can be further adjusted to provide sufficient levels of the active ingredient or to maintain the desired effect. Additional factors which may be taken into account include the severity of the disease state, age, weight and gender of the patient; diet, desired duration of treatment, method of administration, time and frequency of administration, drug combination(s), reaction sensitivities, and tolerance/response to therapy and the judgment of the treating physician.

A treatment regimen can entail daily administration (e.g., once, twice, thrice, etc. daily), every other day (e.g., once, twice, thrice, etc. every other day), semi-weekly, weekly, once every two weeks, once a month, etc. In one embodiment, treatment can be given as a continuous infusion. Unit doses can be administered on multiple occasions. Intervals can also be irregular as indicated by monitoring clinical symptoms. Alternatively, the unit dose can be administered as a sustained release formulation, in which case less frequent administration is required. Dosage and frequency may vary depending on the patient. It will be understood by one of skill in the art that such guidelines will be adjusted for localized administration, e.g. intranasal, inhalation, rectal, etc., or for systemic administration, e.g. oral, rectal (e.g., via enema), i.m. (intramuscular), i.p. (intraperitoneal), i.v. (intravenous), s.c. (subcutaneous), transurethral, and the like.

In some embodiments, the present lysins or active fragments thereof or variants or derivatives thereof are administered to a subject in need thereof in MIC quantities. As is known in the art, a MIC value refers to the minimum concentration of peptide sufficient to suppress at least 80% of the bacterial growth compared to control. Without being limited by theory, it is believed that the present lysins or active fragments thereof or variants or derivatives thereof when administered at MIC levels or higher may be effective against infective endocarditis when co-administered with one or more conventional antibiotics and can exhibit a bacteriocidal effect against a broad range of Gram-positive bacteria including S. aureus as described herein. In addition, in some embodiments, administration of the present lysins or active fragments thereof or variants or derivatives thereof at MIC levels or higher may be used to eradicate biofilms in the subject.

The MIC may be determined by any suitable method. For example, MIC values may be determined using the broth microdilution method according to the Clinical and Laboratory Standards Institute methodology (CLSI), 2018, Methods for Dilution Antimicrobial Susceptibility Tests for Bacteria That Grow Aerobically, 11^{th} Edition, Clinical and Laboratory Standards Institute, Wayne, Pa. In some embodiments, the MIC values for the lysins or active fragments thereof or variants or derivatives thereof are tested using 100% human serum or cation adjusted Mueller Hinton II Broth supplemented with horse serum to a final concentration of 25% and dithiothreitol (DTT) to a final concentration of 0.5 mM to determine a MIC value suitable for in vivo environments.

In some embodiments, the lysins or active fragments thereof or variants or derivatives thereof may also be efficaciously used in the treatment or prevention of infective endocarditis including a recurrence thereof by administering such biologics at sub-MIC levels, e.g., at sub-MIC levels ranging from 0.9× MIC to 0.0001× MIC. At such sub-MIC levels, the present lysins or active fragments thereof or variants or derivatives thereof are typically used to inhibit the growth of Gram-positive bacteria, reduce agglutination, and/or inhibit biofilm formation or to reduce or eradicate biofilm.

Without being limited by theory, sub-MIC dosages of the present lysins or active fragments thereof or variants or derivatives thereof result in non-lethal damage to the cell envelope, mediated by peptidoglycan hydrolytic activity of the lysins or active fragments thereof or variants or derivatives thereof. In some embodiments, the resulting physical and functional changes in the cell envelope account for growth delays. Such physical and functional changes include e.g., destabilization of the cell wall, increases in membrane permeability and dissipation of membrane potential. Although the present lysins or active fragments thereof or variants or derivatives thereof do not directly act on the bacterial cell membrane, any effects on cell membrane permeability and electrostatic potential are likely the result of osmotic stress induced by the peptidoglycan hydrolytic activity of lysin (and destabilization of the cell envelope) at very low concentrations. It is also postulated that localized cell wall hydrolysis can result in the extrusion of inner membrane and the formation of pores as well as the uncoupling of cell synthesis and hydrolysis, changes in cell wall thickness resulting in subsequent growth arrest.

In some embodiments, the sub-MIC concentrations of the present lysins or active fragments thereof or variants or derivatives thereof damage the bacterial cell envelope resulting in bacteria that are more susceptible to conventional antibiotics than in the absence of the sub-MIC dose of the present lysins or active fragments thereof or variants or derivatives thereof.

In some embodiments, the efficacy of sub-MIC level of the present lysins or active fragments thereof or variants or derivatives thereof may be determined using in vitro pharmacodynamic (PD) parameters, as described, for example, in the poster presentation at the American Society for Microbiology (ASM) Microbe on Jun. 2, 2017 in New Orleans, La. by Jun Oh and Raymond Schuch entitled “The Sub-MIC Effect of Lysin CF-301 on Staphylococcus aureus (S. aureus).” See also the world wide web at contrafect.com/technology/publications-posters?page=2. The foregoing described poster presentation is herein incorporated by reference in its entirety.

Briefly, in vitro pharmacodynamic (PD) parameters including the postantibiotic effect (PAE), PA sub-MIC effect (PA-SME) and sub-MIC effect (SME), allow for a determination of the impact of short-duration and/or sub-MIC exposures on bacterial growth. By definition, the PAE is a suppressed phase of bacterial growth that persists after initial exposure to an antimicrobial agent (often at supra-MIC levels) until normal bacterial growth resumes after removal of the antibacterial agent. The PA-SME is suppressed growth during exposure to sub-MICs in the PAE phase; the PA-SME, thus, represents the time interval that includes PAE plus the additional time during which growth is suppressed by sub-MICs. Since sub-inhibitory concentrations may exist after dosing in therapeutic settings, the PA-SME can reflect the in vivo situation more closely than the PAE. In contrast to the PA-SME, the SME measures the impact of sub-inhibitory levels on the growth of bacteria which have not been previously exposed to e.g. a lysin or antibiotic.

The in vitro PAE may be determined by subjecting Gram-positive bacteria cultures to a lysin of interest at, for example, 4× the MIC for e.g., 1 hour at 37° C. with agitation. Following exposure, the lysin is removed by e.g., 1:1,000 dilution into freshly prepared media and then further incubated at 37° C. with agitation at 200 rpm for 24 hours. For each PAE test culture, bacterial concentrations are determined by quantitative plating just before and immediately after dilution; growth can then be followed by quantitative plating at e.g., one hour intervals for e.g. 24 hours. The PAE is defined as T-C; where T is the time required for viability counts of an antibiotic- or lysin-exposed culture to increase by 1-log₁₀ above counts immediately after removal of lysin and C is the corresponding time for growth control not exposed to lysin.

The in vitro PA-SME may be determined as follows. After PAE induction for 1 hour with lysin, culture samples are diluted e.g., 1:1,000 into aliquots of medium containing four different sub-MIC concentrations of lysin and further incubated at 37° C. with agitation at 225 rpm for 24 hours. Viability may be determined as described above for in vitro PAE determination. The PA-SME is defined as Tₚₐ-C; where Tₚₐ is the time required for cultures previously exposed to lysin and then exposed to different sub-MIC concentrations to increase by 1-log₁₀ above counts immediately after the removal of lysin and C is the corresponding time for the growth control not exposed to lysin.

The in vitro SME may be induced the same way as the PA-SME, without the prior induction of the PAE. Following a 1 hour growth phase (without lysin), cultures samples are diluted 1:1,000 into 100% human serum containing different sub-MIC concentrations of lysin and then further incubated at 37° C. with agitation at 225 rpm for up to 24 hours. Viability counts are determined as above for the in vitro PAE determination. The SME is defined as Tₛ-C; where Tₛ is the time required for the cultures exposed only to sub-MIC concentrations to increase 1-log₁₀ above counts immediately after dilution; C is the corresponding time for the unexposed control.

In some embodiments, the efficacy of the sub-MIC value of a lysin or active fragment thereof or variant or derivative thereof may be assessed by determining an in vivo PA-SME value using, e.g., the neutropenic mouse thigh model. This model tests for Gram-positive bacteria regrowth inhibition after lysin levels fall below the MIC and is considered to primarily provide a description of the sub-MIC effect that is further influenced by in vivo infection conditions including in vivo biofilm formation. The PA-SME may be determined using the following equation PAE=T-C-M, where M represents the time for which serum levels exceed the MIC, T is the time required for CFUs in the thighs, of the treated mouse to increase 1-log₁₀ above the count at time M, and C is the time needed for CFUs in the thighs of untreated controls to increase 1-log₁₀ above the viable counts at T=0 hour.

In some embodiments, the present lysin or active fragment thereof or variant or derivative thereof at sub-MIC and/or MIC level doses are capable of reducing a biofilm, in particular an in vivo biofilm. As is known in the art, in vivo biofilms may be structurally distinct from in vitro biofilms. Typically, the reason for the differences between in vitro biofilms and in vivo biofilms, such as those associated with chronic infections, is the lack of defense mechanism exposure in in vitro biofilm systems. In most in vivo biofilm habitats, phagocytes, and even bacteriophages may be present, along with the presence of pus and other excreted fluids and polymers. Such variables are generally avoided in in vitro model systems where they are difficult to control or reproduce. In some embodiments, the present methods are advantageously used to eradicate or reduce the more structurally complex in vivo biofilms.

In some embodiments, the present lysins or active fragments thereof or variants or derivatives thereof reduce the MIC of an antibiotic needed for bacteriocidal and/or bacteriostatic activity. Any known method to assess the MIC may be used. In some embodiments, a checkerboard assay is used to determine the effect of a lysin on antibiotic concentration. The checkerboard assay is based on a modification of the CLSI method for MIC determination by broth microdilution as described herein.

Checkerboards are constructed by first preparing columns of e.g., a 96-well polypropylene microtiter plate, wherein each well has the same amount of antibiotic diluted 2-fold along the horizontal axis. In a separate plate, comparable rows are prepared in which each well has the same amount of lysin diluted e.g., 2-fold along the vertical axis. The lysin and antibiotic dilutions are then combined, so that each column has a constant amount of antibiotic and doubling dilutions of lysin, while each row has a constant amount of lysin and doubling dilutions of antibiotic. Each well thus has a unique combination of lysin and antibiotic. Bacteria are added to the drug combinations at concentrations of 1×10⁵ CFU/ml in e.g., cation adjusted Mueller Hinton II Broth supplemented with horse serum to a final concentration of 25% and dithiothreitol (DTT) to a final concentration of 0.5 mM, for example. The MIC of each drug, alone and in combination, is then recorded after e.g., 16 hours at 37° C. in ambient air. Summation fractional inhibitory concentrations (ΣFICs) are calculated for each drug and the minimum ΣFIC value (ΣFICmin) is used to determine the effect of the lysin/antibiotic combination.

In some embodiments, the one or more antibiotics of the present disclosure are administered to a subject in need thereof at the MIC level or greater than the MIC level, such as 1× MIC, 2× MIC, 3× MIC and 4× MIC. In other embodiments, the antibiotics are administered at a sub-MIC level, e.g., ranging from 0.9× MIC to 0.0001× MIC.

In some embodiments, the present lysins or active fragments thereof or variants or derivatives thereof and the one or more antibiotics of the present method, such as daptomycin, are administered simultaneously. In other embodiments, the present lysins or active fragments thereof or variants or derivatives thereof and the one or more antibiotics of the present method, such as daptomycin, are administered in series, such as sequentially, in any order. In some embodiments, the lysin is administered during or subsequent to administration of a standard of care antibiotic treatment, e.g., a two-week course of oxacillin and gentamicin or daptomycin. The present lysins or active fragments thereof or variants or derivatives thereof and the present one or more antibiotics may be administered in a single dose or multiple doses, singly or in combination.

The lysins or active fragments thereof or variants or derivatives thereof and the one or more antibiotics of the present disclosure may be administered by the same mode of administration or by different modes of administration, and may be administered once, twice or multiple times, one or more in combination or individually. Thus, the present lysins or active fragments thereof or variants or derivatives thereof may be administered in an initial dose followed by a subsequent dose or doses, particularly depending on the response, e.g., the bacteriocidal and/or bacteriostatic effects and/or the effect on agglutination and/or biofilm formation or reduction, and may be combined or alternated with antibiotic dose(s). Typically, the lysins or active fragments thereof or variants or derivatives thereof are administered in a single bolus followed by conventional doses and administration modes of the one or more antibiotics of the present disclosure.

In more typical embodiments, a single bolus of a lysin or active fragment thereof or variant or derivative thereof of the present disclosure is administered to a subject followed by a conventional regimen, e.g., standard of care (SOC) dosages, of one or more antibiotics of the present disclosure, such as daptomycin. In other typical embodiments, one or more antibiotics of the present disclosure, such as daptomycin, is administered to a subject followed by a single bolus of a lysin or active fragment thereof or variant or derivative thereof of the present disclosure, followed by additional dosages of the one or more antibiotics of the present disclosure at conventional dosages, such as daptomycin. Even more typically, a single sub-MIC dose of the lysin or active fragment thereof or variant or derivative thereof is administered to a subject followed by a conventional regimen of one or more doses of the one or more antibiotics of the present disclosure. In other, even more typical embodiments, one or more antibiotics of the present disclosure such as daptomycin is administered to a subject at a conventional dosage followed by a single bolus at a sub-MIC dose of lysin or active fragment thereof or variant or derivative thereof of the present disclosure, followed by additional dosages of the one or more antibiotics of the present disclosure at conventional dosages, such as daptomycin.

In other embodiments, a single sub-MIC dose of the lysin or active fragment thereof or variant or derivative thereof of the present disclosure is administered to a subject followed by one or more doses of the one or more antibiotics of the present disclosure, such as daptomycin, wherein the antibiotic dose(s) is also administered at a sub-MIC level.

In other embodiments, one or more antibiotics of the present disclosure such as daptomycin is administered to a subject at a sub-MIC dosage followed by a single bolus at a sub-MIC dosage of a lysin or active fragment thereof or variant or derivative thereof of the present disclosure, followed by one or more additional dosages of the one or more antibiotics of the present disclosure at sub-MIC dosages, such as daptomycin.

Formulations

The lysin or active fragment thereof or variant or derivatives thereof of the present disclosure may be administered with the one or more antibiotics described herein. The lysin or active fragment thereof or variant or derivatives thereof and antibiotics may each be included in a single pharmaceutical formulation or be separately formulated in the form of a solution, a suspension, an emulsion, an inhalable powder, an aerosol, or a spray, tablets, pills, pellets, capsules, capsules containing liquids, powders, sustained-release formulations, suppositories, tampon applications emulsions, aerosols, sprays, suspensions, lozenges, troches, candies, injectants, chewing gums, ointments, smears, time-release patches, liquid absorbed wipes, and combinations thereof.

In some embodiments, administration of the pharmaceutical formulations may include systemic administration. Systemic administration can be enteral or oral, i.e., a substance is given via the digestive tract, parenteral, i.e., a substance is given by other routes than the digestive tract such as by injection or inhalation. Thus, the lysins or active fragments thereof or variants or derivatives thereof and/or the one or more antibiotics of the present disclosure can be administered to a subject orally, parenterally, by inhalation, topically, rectally, nasally, buccally or via an implanted reservoir or by any other known method. The lysins or active fragments thereof or variants or derivatives thereof and/or the one or more antibiotics of the present disclosure can also be administered by means of sustained release dosage forms.

For oral administration, the lysins or active fragments thereof or variants or derivatives thereof and/or the one or more antibiotics of the present disclosure can be formulated into solid or liquid preparations, for example tablets, capsules, powders, solutions, suspensions and dispersions. In some embodiments, the lysins or active fragments thereof or variants or derivatives thereof and/or the one or more antibiotics of the present disclosure can be formulated with excipients such as, e.g., lactose, sucrose, corn starch, gelatin, potato starch, alginic acid and/or magnesium stearate.

For preparing solid compositions such as tablets and pills, the lysins or active fragments thereof or variants or derivatives thereof and/or the one or more antibiotics of the present disclosure is mixed with a pharmaceutical excipient to form a solid pre-formulation composition. If desired, tablets may be sugar coated or enteric coated by standard techniques. The tablets or pills may be coated or otherwise compounded to provide a dosage form affording the advantage of prolonged action. For example, the tablet or pill can include an inner dosage and an outer dosage component, the latter being in the form of an envelope over the former. The two dosage components can be separated by an enteric layer, which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

In another embodiment, the pharmaceutical formulations of the present disclosure are formulated as inhalable compositions. In some embodiments, the present pharmaceutical formulations are advantageously formulated as a dry, inhalable powder. In specific embodiments, the present pharmaceutical formulations may further be formulated with a propellant for aerosol delivery. Examples of suitable propellants include, but are not limited to: dichlorodifluoromethane, trichlorofluoromethane, dichloro-tetrafluoroethane and carbon dioxide. In certain embodiments, the formulations may be nebulized.

In some embodiments, the inhalable pharmaceutical formulations include excipients. Examples of suitable excipients include, but are not limited to: lactose, starch, propylene glycol diesters of medium chain fatty acids; triglyceride esters of medium chain fatty acids, short chains, or long chains, or any combination thereof; perfluorodimethylcyclobutane; perfluorocyclobutane; polyethylene glycol; menthol; lauroglycol; diethylene glycol monoethylether; polyglycolized glycerides of medium chain fatty acids; alcohols; eucalyptus oil; short chain fatty acids; and combinations thereof.

A surfactant can be added to an inhalable pharmaceutical formulation of the present disclosure in order to lower the surface and interfacial tension between the medicaments and the propellant. The surfactant may be any suitable, non-toxic compound which is non-reactive with the present polypeptides. Examples of suitable surfactants include, but are not limited to: oleic acid; sorbitan trioleate; cetyl pyridinium chloride; soya lecithin; polyoxyethylene(20) sorbitan monolaurate; polyoxyethylene (10) stearyl ether; polyoxyethylene (2) oleyl ether; polyoxypropylene-polyoxyethylene ethylene diamine block copolymers; polyoxyethylene(20) sorbitan monostearate; polyoxyethylene(20) sorbitan monooleate; polyoxypropylene-polyoxyethylene block copolymers; castor oil ethoxylate; and combinations thereof.

In some embodiments, the pharmaceutical formulations of the present disclosure comprise nasal formulations. Nasal formulations include, for instance, nasal sprays, nasal drops, nasal ointments, nasal washes, nasal injections, nasal packings, bronchial sprays and inhalers, or indirectly through use of throat lozenges, mouthwashes or gargles, or through the use of ointments applied to the nasal nares, or the face or any combination of these and similar methods of application.

The pharmaceutical formulations of the present disclosure are more typically administered by injection. For example, the pharmaceutical formulations can be administered intramuscularly, intrathecally, subdermally, subcutaneously, or intravenously to treat infections by Gram-positive bacteria, typically, infective endocarditis caused by S. aureus, including methicillin-resistant S. aureus (MRSA). The pharmaceutically acceptable carrier may be comprised of distilled water, a saline solution, albumin, a serum, or any combinations thereof. Additionally, pharmaceutical formulations of parenteral injections can comprise pH buffered solutions, adjuvants (e.g., preservatives, wetting agents, emulsifying agents, and dispersing agents), liposomal formulations, nanoparticles, dispersions, suspensions or emulsions as well as sterile powders for reconstitution into sterile injectable solutions or dispersions just prior to use.

In cases where parenteral injection is the chosen mode of administration, an isotonic formulation is typically used. Generally, additives for isotonicity can include sodium chloride, dextrose, mannitol, sorbitol, and lactose. In some cases, isotonic solutions such as phosphate buffered saline are preferred. Stabilizers can include gelatin and albumin. A vasoconstriction agent can be added to the formulation. The pharmaceutical preparations according to this type of application are provided sterile and pyrogen free.

The pharmaceutical formulations of the present disclosure may be presented in unit dosage form and may be prepared by any methods well known in the art. The amount of active ingredients which can be combined with a carrier material to produce a single dosage form will vary depending upon the host being treated, the duration of exposure of the recipient to the infectious bacteria, the size and weight of the subject, and the particular mode of administration. The amount of active ingredients that can be combined with a carrier material to produce a single dosage form will generally be that amount of each compound which produces a therapeutic effect. Generally, out of one hundred percent, the total amount will range from about 1 percent to about ninety-nine percent of active ingredients, typically from about 5 percent to about 70 percent, most typically from about 10 percent to about 30 percent.

### EXAMPLES

### Example 1. In Vitro Efficacy of a Lysin of the Disclosure Against Staphylococcus and Streptococcus Species Associated with Infective Endocarditis

The in vitro activity of CF-301 (exebacase) and comparator antibiotics, e.g. daptomycin and vancomycin, were evaluated against a range of bacterial species most commonly associated with infective endocarditis as described herein and shown in Table 2. A variety of strains and isolates were acquired from collections and repositories in the United States, Europe and Asia. The strains and isolates were confirmed at the species level by each source. The majority of isolates were isolated from a range of infection types, including bacteremia (and endocarditis), skin and soft tissue infections, and respiratory infections. A range of infections types were included to ensure a sufficient number of isolates for each target species.

Minimal inhibitory concentrations (MICs) of exebacase against staphylococci were determined by broth microdilution (BMD) using a nonstandard antimicrobial susceptibility testing (AST) medium comprised of cation-adjusted Mueller Hinton broth (caMHB) supplemented with horse serum (Sigma Aldrich) and dithiothreitol (DTT; Sigma Aldrich) to final concentrations of 25% and 0.5 mM, respectively. This medium, referred to as caMHB-HSD, is approved for use in exebacase AST by the Clinical and Laboratory Standards Institute (CLSI) (CLSI. 2017, Jan. 16-17. AST Subcommittee Working Group Meetings and Plenary. AST Meeting Files & Resources, clsi.org/education/microbiology/ast/ast-meeting-files-resources/. Additional supplementation with 2.5% lysed red blood cells (Remel™, ThermoFisher) was included for analyses of streptococcal isolates, as recommended by CLSI. See CLSI, 2015. Methods for Dilution Antimicrobial Susceptibility Tests for Bacteria That Grow Aerobically, 10th Edition. Clinical and Laboratory Standards Institute, Wayne, Pa.

Daptomycin (Sigma Aldrich) and vancomycin hydrochloride (Sigma Aldrich) were tested following the reference BMD method for each. See CLSI. 2015. Methods for Dilution Antimicrobial Susceptibility Tests for Bacteria That Grow Aerobically, 10th Edition. Clinical and Laboratory Standards Institute, Wayne, Pa.

Exebacase activity was first confirmed using sets of 73 MSSA and 74 MRSA isolates, which demonstrated MIC_{50/90} values of 0.5/0.5 μg/mL and 0.5/1 μg/mL and ranges of 0.25-1 μg/mL and 0.5-2 μg/mL, respectively (Table 3). Similar levels of activity were next observed for each coagulase-negative staphylococcal species, including S. epidermidis (MIC_{50/90}=0.5/0.5 μg/mL), S. lugdunensis (MIC_{50/90}=1/1 μg/mL), S. haemolyticus (MIC_{50/90}=0.5/1 μg/mL), S. capitis (MIC_{50/90}=1/2 μg/mL) and S. warneri (MIC_{50/90}=0.5/1 μg/mL). Staphylococcus hominis, only rarely associated with IE, was tested (n=2 strains) and demonstrated exebacase MIC values of 0.125 μg/mL and 0.25 μg/mL (data not shown). Other staphylococcal species, were also tested, including S. pseudintermedius (MIC=0.25 μg/mL, each of n=6 isolates), S. sciuri (MIC=2 μg/mL, n=3 isolates), S. simulans (MIC=0.125 μg/mL, n=1 isolate), and S. hyicus (MIC=0.25 μg/mL, n=1 isolate). MICs for daptomycin and vancomycin were observed with ranges of 0.125-2 μg/mL and 0.5-4 μg/mL, respectively, for all staphylococci tested, consistent with expected ranges. See Sader et al., 2019, J. Antimicrob. Chemother. doi:10.1093/jac/dkz006 and Pfaller et al., 2018, Int. J. Antimicrob. Agents. 51:608-611.

The majority of viridans streptococci tested, in addition to S. pneumoniae and E. faecalis (formerly Group D Streptococcus), exhibited highly variable and low level susceptibilities to exebacase, with MIC values ranging as high as 8 to greater than 512 μg/mL (Table 4). Notable exceptions included S. intermedius, S. pyogenes (Lancefield group A), S. agalactiae (Lancefield group B) and S. dysgalactiae (Lancefield group G), with MIC ranges of 0.06-0.5 μg/mL, 0.5-4 μg/mL, 0.25-4 μg/mL, and 1-2 μg/mL, respectively. Unlike many of the viridans streptococci and E. faecalis which primarily cause subacute IE, S. intermedius (a viridans group species) and both S. agalactiae and S. dysgalactiae are associated with the more aggressive acute disease caused by staphylococci and known in the art to result in rapid destruction of the endocardium.

Overall, the data presented here demonstrated the potent in vitro activity of exebacase against all staphylococcal species and a subset of streptococci including those associated with acute IE. These findings are particularly significant considering the increasing incidence of staphylococcal IE infections and the decreasing incidence of infections associated with viridans group streptococci.

TABLE 2
Review of data from 7 studies examining the causative agents of infective endocarditis in humans

| | Microorganisms identified by blood culture (%)^{a} | | | | | | |
|---|---|---|---|---|---|---|---|
| | (1a) | (1b) | (1c) | (1d) | (1e) | (1f) | (1g) |
| Organism | N = 167 | N = 2781 | N = 360 | N = 1804 | N = 105 | N = 497 | N = 212 |
| Staphylococcus aureus | 44.3 | 31.0 | 24.7 | 40.3 | 10.4 | 26.6 | 23.6 |
| Staphylococcus epidermidis | 1.8 | | 6.4 | | | | 6.1 |
| Staphylococcus lugdunensis | 1.8 | | 1.4 | | | | 0.9 |
| Other CoNS^{b} | 3.0 | 11.0^{e} | 4.6 | 16.7^{f} | 12.4^{f} | 9.7^{f} | 5.3 |
| Streptococcus viridans^{c} | 6.6 | 17.0 | 38.6 | 12.3 | 58.1 | | 16.0 |
| Streptococcus agalactiae | 3.0 | | 1.4 | | | | 7.4 |
| Streptococcus pyogenes | | | | | | | 0.9 |
| Streptococcus pneumoniae | | | 0.6 | | | | |
| Streptococcus gallolyticus | | 6 | 6.1 | 6.4 | | 12.5 | 7.1 |
| “oral” streptococci^{d} | | | | | | 18.7 | |
| Streptococcus group G | | | | | | | 1.4 |
| Enterococcus faecalis | 6.6 | | 11.1 | | 4.8 | | 11.8 |
| Enterococcus spp.^{e} | | | | 12.7 | | | |

^{a}References for each study are indicated as follows (N = # of patients in each study). 1a. Yuan SM, 2014, Int. J. Clin. Exp. Med. 7: 199-218, 1b. Murdoch et al. 2009, Arch. Intern. Med. 169: 463-73, 1c. Farag et al., 2017, Med. Sci. Monit. 23: 3617-3626, 1d. Munoz et al. Spanish Collaboration on Endocarditis-Grupo de Apoyo al Manejo de la Endocarditis le. Infecciosa en E. 2015. Current Epidemiology and Outcome of Infective Endocarditis: A Multicenter, Prospective, Cohort Study. Medicine (Baltimore) 94: e1816, 1e. Xu H. et al., 2016. PLoS One 11: e0166764, 1f. Selton-Set al. 2012, Clin. Infect. Dis. 54: 1230-9, 1g. Yombi et al., 2017, Acta. Clin. Belg. 72: 417-423.
^{b}Some studies here distinguish S. epidermidis and S. lugdunensis from other more infrequent CoNS organisms associated with IE including S. capitis, S. warneri, and S. haemolyticus (Petti et al., 2008, J. Clin. Microbiol. 46: 1780-4, Farag et al., 2017, Med. Sci. Monit. 23: 3617-3626 and Kuvhenguhwa et al., 2017. Cardiol. Res. 8: 236-240).
^{c}The Viridans Group Streptococci causing IE include: S. mitis, S. sanguinis, S. mutans, S. salivarius, S. gordonii, S. intermdius and S. anginosus. See Cunha et al., 2010, Heart Lung 39: 64-72, Kim et al., 2018, Diagn. Microbiol. Infect. Dis., 91: 269-272, Naveen et al., 2014, Int. J. Med. Microbiol., 304: 262-8 and Dadon et al., 2017, Ann. Clin. Microbiol. Antimicrob., 16: 68.
^{d}Viridans streptococci are referred to as oral streptococci in the indicated study.
^{e}Species not provided, however, E. faecalis causes about 97% of IE cases associated with enterococci. See Baddour et al., 2015, Circulation, 132: 1435-86.
^{f}These studies group all CoNS species together.

TABLE 3
Susceptibility of Staphylococcus species to exebacase and comparator antibiotics^{a}

| | | CF-301 | | | | DAP | VAN | | | |
|---|---|---|---|---|---|---|---|---|---|---|
| Organism | N | MIC₅₀^{a} | MIC₉₀ | Range | MIC₅₀ | MIC₉₀ | Range | MIC₅₀ | MIC₉₀ | Range |
| S. aureus (MSSA) | 73 | 0.5 | 0.5 | 0.25-1 | 0.25 | 0.25 | 0.125-0.5 | 1 | 1 | 0.5-1 |
| S. aureus (MRSA) | 74 | 0.5 | 1 | 0.5-2 | 0.25 | 0.5 | 0.125-1 | 1 | 1 | 1-2 |
| S. epidermidis^{b} | 52 | 0.5 | 0.5 | 0.125-2 | n.d. | n.d. | n.d. | n.d. | n.d. | n.d. |
| S. lugdunensis | 49 | 1 | 1 | 0.25-2 | 0.5 | 1 | 0.25-2 | 1 | 1 | 0.5-2 |
| S. haemolyticus | 36 | 0.5 | 1 | 0.25-2 | 0.5 | 1 | 0.25-2 | 1 | 2 | 0.5-4 |
| S. capitis | 13 | 1 | 2 | 0.25-4 | 0.5 | 1 | 0.25-1 | 1 | 1 | 0.5-2 |
| S. warneri | 23 | 0.5 | 1 | 0.06-1 | 0.5 | 2 | 0.25-2 | 1 | 2 | 0.25-2 |

^{a}MIC values are indicated in μg/mL
^{b}MIC values for DAP and VAN data were not determined (n.d.) for S. epidermidis.

TABLE 4
Susceptibility of Streptococcus and Enterococcus species to exebacase and comparator antibiotics^{a}

| | Streptococcus | | CF-301 | | | | DAP | VAN | | | |
|---|---|---|---|---|---|---|---|---|---|---|---|
| Organism | Group | N | MIC₅₀^{a} | MIC₉₀ | Range | MIC₅₀ | MIC₉₀ | Range | MIC₅₀ | MIC₉₀ | Range |
| S. anginosus | viridans | 10 | 32 | 64 | 1-64 | 0.5 | 0.5 | 0.25-0.5 | 0.5 | 1 | 0.5-1 |
| S. gordonii | viridans | 11 | 4 | 8 | 0.5-8 | 0.5 | 0.5 | 0.25-1 | 0.5 | 1 | 0.5-1 |
| S. mitis | viridans | 18 | 2 | 8 | 0.5-64 | 0.5 | 1 | 0.125-1 | 0.5 | 0.5 | 0.25-0.5 |
| S. mutans | viridans | 22 | 32 | 64 | 1->64 | 0.5 | 1 | 0.25-8 | 1 | 1 | 0.25-1 |
| S. oralis | viridans | 15 | 4 | 64 | 0.5-64 | 0.5 | 0.5 | 0.25-1 | 0.5 | 1 | 0.5-1 |
| S. salivarius | viridans | 12 | 2 | 8 | 0.5-8 | 0.25 | 0.5 | 0.06-0.5 | 0.5 | 0.5 | 0.25-0.5 |
| S. sanguinis | viridans | 15 | 4 | 16 | 2-32 | 0.25 | 1 | 0.06-1 | 0.5 | 0.5 | 0.5-2 |
| S. intermedius^{b} | viridans | 10 | 0.25 | 0.25 | 0.06-0.5 | n.d | n.d | n.d | n.d | n.d | n.d |
| S. gallolyticus | bovis | 19 | 64 | >512 | 0.25->512 | 0.125 | 0.25 | 0.06-0.5 | 0.25 | 0.5 | 0.25-0.5 |
| S. pyogenes | A | 100 | 1 | 2 | 0.5-4 | 0.03 | 0.06 | 0.016-0.06 | 0.5 | 0.5 | 0.25-0.5 |
| S. agalactiae | B | 97 | 1 | 2 | 0.25-4 | 0.125 | 0.25 | 0.125-0.25 | 0.5 | 0.5 | 0.25-0.5 |
| S. dysgalactiae | G | 22 | 1 | 2 | 1-2 | 0.125 | 0.25 | 0.06-0.5 | 0.5 | 0.5 | 0.25-1 |
| S. pneumoniae | | 59 | 4 | 32 | 1-64 | 0.125 | 0.5 | 0.06-0.25 | 0.25 | 0.5 | 0.25-0.5 |
| E. faecalis | D (formerly) | 18 | 16 | 64 | 1-256 | 0.5 | 0.5 | 0.25-0.5 | 1 | 2 | 0.5-2 |

^{a}MIC values are indicated in μg/mL
^{b}MIC values for DAP and VAN data were not determined (n.d.) for S. intermedius.

### Example 2. Effect of CF-301 Administration in Series with Daptomycin in an Infective Endocarditis Rabbit Model

Materials and Methods

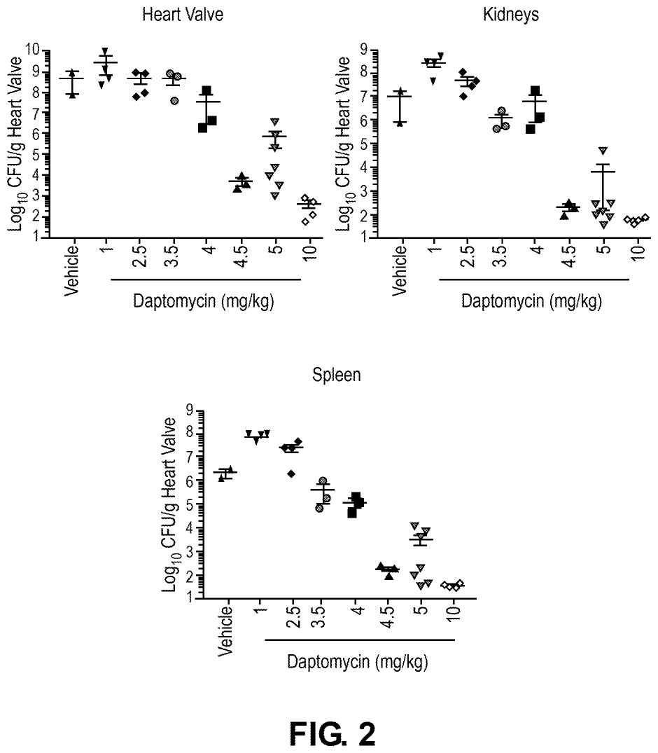

The in vivo efficacy of PlySs2 (CF-301) against a classic S. aureus “biofilm” infection model was evaluated in the presence of daptomycin doses below the human therapeutic dose (HTD)-equivalent. The rational for selection of the daptomycin dose is as follows. Daptomycin pilot dose-response experiments were performed over a range from 1 mg/kg to 10 mg/kg, administered intravenously, once daily for 4 days in the infective endocarditis rabbit model described below, which was caused by the MRSA strain, MW2. FIG. 2 depicts data for individual animals, plotted as treatment regimen versus log₁₀ CFU/g tissue (mean±SEM are shown). From these studies, a daptomycin dose-response was defined. Daptomycin at 4 mg/kg, a dose below the HTD equivalent, was chosen to explore a synergistic benefit of CF-301 therapy in addition to daptomycin. In the rabbit infective endocarditis model, a daptomycin-alone dose of 4 mg/kg administered intravenously provided about 0.25 to 1.45 log₁₀ reduction in bacterial burden compared to vehicle-treated controls. Treated animals still had burdens of about 5-7 log₁₀, providing a dynamic range over which to observe the potential added effects of CF-301 in this treatment regimen.

A well-described indwelling transcarotid artery-to-left ventricle catheter-induced model of aortic valve infective endocarditis was used in rabbits. See Xiong et al., 2011, AAC, 55:P5325-5330. At 48 hours after catheter placement, infective endocarditis was induced by intravenous inoculums of about 2×10⁵ CFU (the induction of ID95 of MRSA strain MW2 in this model). At 24 hours post-infection, animals were randomized into seven groups: i) Buffer controls; or ii)-iv) CF-301 (a sub-MIC dose of 0.09 mg/kg) administered as a single intravenous dose (5-10 minutes infusion) at 1 or 4 hours prior to daptomycin administration versus immediately post-daptomycin administration or 2 or 4 hours post-daptomycin administration (4 mg/kg intravenous). Daptomycin administration was continued once a day for 4 days. At 24 hours after the last dose of daptomycin, animals were humanely euthanized and cardiac vegetations, kidneys, and spleens were sterilely removed and quantitatively cultured. Bacterial density for each organ for the different treatment groups were calculated as mean log₁₀ CFU/g of tissue (±95% confidence interval).

Results

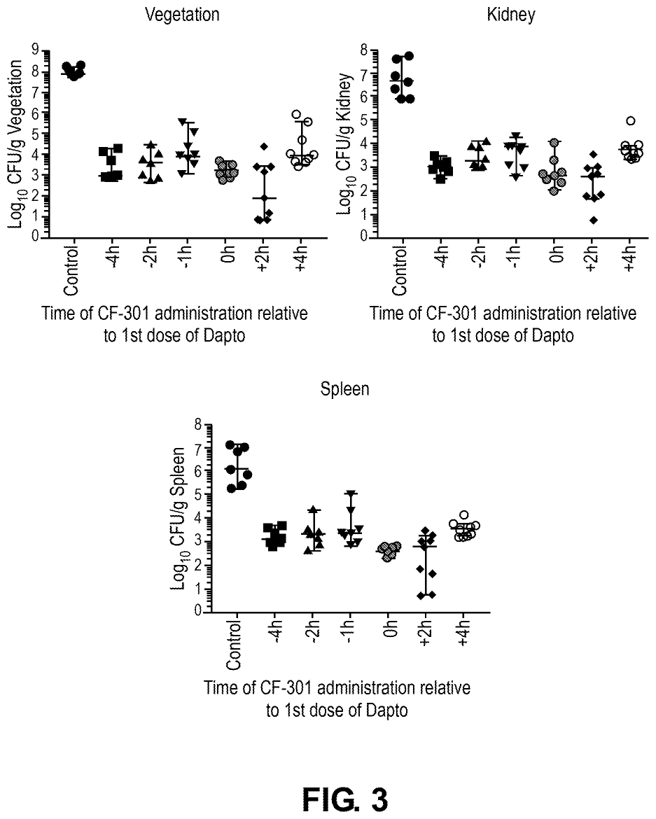

The addition of a single dose of CF-301 to daptomycin regimen at all time-points tested (either before or after the initiation of treatment with daptomycin) significantly reduced MRSA densities in all three target tissues as compared to the controls and daptomycin alone (FIG. 3 and Table 3). No statistically significant differences were observed for any group treated with the combination of CF-301 and daptomycin (Table 4).

TABLE 3
MRSA Densities in Target Tissues

| | Rabbits | Mean Log10 CFU/g tissue ± STD | | |
|---|---|---|---|---|
| Groups | (N/group) | Vegetation | Kidneys | Spleen |
| Vehicle Control | 7 | 8.05 ± 0.19 | 6.70 ± 0.76 | 6.23 ± 0.79 |
| CF-301 (0.09 mg/kg) administered 4 hours | 7 | 3.45 ± 0.63 | 2.97 ± 0.32 | 3.18 ± 0.35 |
| prior to the initial dose of DAP 4 mg/kg; | | | | |
| IV QD x 4 days | | | | |
| CF-301 (0.09 mg/kg) administered 2 hours | 7 | 3.54 ± 0.68 | 3.48 ± 0.47 | 3.31 ± 0.57 |
| prior to the initial dose of DAP 4 mg/kg; | | | | |
| IV QD x 4 days | | | | |
| CF-301 (0.09 mg/kg) administered 1 hour | 8 | 4.16 ± 0.82 | 3.53 ± 0.59 | 3.55 ± 0.74 |
| prior to the initial dose of DAP 4 mg/kg; | | | | |
| IV QD x 4 days | | | | |
| CF-301 (0.09 mg/kg) administered immediately | 8 | 3.23 ± 0.36 | 2.77 ± 0.65 | 2.60 ± 0.17 |
| after the initial dose of DAP 4 mg/kg; | | | | |
| IV QD x 4 days | | | | |
| CF-301 (0.09 mg/kg) administered 2 hours | 9 | 2.25 ± 1.39 | 2.29 ± 0.87 | 2.23 ± 1.09 |
| after the initial dose of DAP 4 mg/kg; | | | | |
| IV QD x 4 days | | | | |
| CF-301 (0.09 mg/kg) administered 4 hours | 9 | 4.31 ± 0.89 | 3.78 ± 0.51 | 3.49 ± 0.33 |
| after the initial dose of DAP 4 mg/kg; | | | | |
| IV QD x 4 days | | | | |

TABLE 4
Statistical Comparison of Treatment Groups*

| | | Calculated P Value | | |
|---|---|---|---|---|
| | Comparator | Heart Valve | | |
| Groups | Group | Vegetation | Kidneys | Spleen |
| CF-301 (0.09 | Vehicle | <0.001 | <0.001 | <0.001 |
| mg/kg) | 2 hours prior | N.S. | <0.05 | N.S. |
| administered 4 | 1 hour prior | <0.05 | <0.0449 | N.S. |
| hours prior to the | Immediately | N.S. | N.S. | <0.001 |
| initial dose of | post | | | |
| DAP 4 mg/kg; | 2 hours post | N.S. | <0.05 | N.S. |
| IV QD x 4 days | 4 hours post | N.S. | <0.001 | <0.05 |
| CF-301 (0.09 | Vehicle | <0.001 | <0.001 | <0.001 |
| mg/kg) | 4 hours prior | N.S. | <0.05 | N.S. |
| administered 2 | 1 hour prior | <0.05 | <0.05 | N.S. |
| hours prior to the | Immediately | N.S. | N.S. | <0.05 |
| initial dose of | post | | | |
| DAP 4 mg/kg; | 2 hours post | N.S. | <0.05 | <0.05 |
| IV QD x 4 days | 4 hours post | N.S. | <0.00 | N.S. |
| CF-301 (0.09 | Vehicle | <0.001 | <0.001 | <0.001 |
| mg/kg) | 4 hours prior | <0.05 | <0.05 | N.S. |
| administered 1 | 2 hours prior | N.S. | N.S. | N.S. |
| hours prior to the | Immediately | <0.01 | <0.05 | <0.0001 |
| initial dose of | post | | | |
| DAP 4 mg/kg; | 2 hours post | <0.01 | <0.01 | <0.01 |
| IV QD x 4 days | 4 hours post | N.S. | N.S. | N.S. |
| CF-301 (0.09 | Vehicle | <0.002 | <0.0002 | <0.0002 |
| mg/kg) | 4 hours prior | N.S. | N.S. | <0.001 |
| administered | 2 hours prior | N.S. | <0.0200 | <0.01 |
| immediately post | 1 hour prior | <0.01 | <0.05 | <0.0001 |
| the initial dose | 2 hours post | N.S. | <0.01 | N.S. |
| of DAP 4 mg/kg; | 4 hours post | N.S. | N.S. | <0.0001 |
| IV QD x 4 days | | | | |
| CF-301 (0.09 | Vehicle | <0.0001 | <0.0001 | <0.0001 |
| mg/kg) | 4 hours prior | N.S. | <0.05 | N.S. |
| administered 2 | 2 hours prior | <0.05 | <0.0017 | <0.05 |
| hours after the | 1 hour prior | <0.01 | <0.01 | <0.01 |
| initial dose of | Immediately | N.S. | N.S. | N.S. |
| DAP 4 mg/kg; | post | | | |
| IV QD x 4 days | 4 hours post | N.S. | <0.001 | <0.001 |
| CF-301 (0.09 | Vehicle | <0.0001 | <0.0001 | <0.0001 |
| mg/kg) | 4 hours prior | N.S. | <0.0006 | 0.0454 |
| administered 4 | 2 hours prior | N.S. | N.S. | 0.2039 |
| hours after the | 1 hour prior | N.S. | N.S. | N.S. |
| initial dose of | Immediately | <0.05 | <0.01 | <0.0001 |
| DAP 4 mg/kg; | post | | | |
| IV QD x 4 days | 2 hours post | <0.001 | <0.001 | <0.001 |

*Data were analyzed by Student T-test using GraphPad Prism and ranked as non-significant (NS) with a P value greater than 0.05, statistically significant with P values of <0.05 to 0.001.

These results demonstrate that the addition of a single dose of CF-301 to daptomycin at various time points (before daptomycin versus same-time as daptomycin and post-daptomycin dose up to 4 hours) significantly reduced MRSA densities within all relevant target tissues in this model. Surprisingly, these results indicate that co-administering CF-301 and daptomycin can be used to effectively treat MRSA in the context of an in vivo biofilm environment. These data also suggest there is a relatively wide time-window for optimal and efficacious administration of CF-301 relative to dosing of conventional anti-staphylococcal antibiotics, such as daptomycin.

### Example 3. CF-301 and Background Standard of Care (SOC) Antibacterial Therapy for the Treatment of S. aureus Bacteremia, Including Endocarditis in Adults

Materials and Methods

Seventy-one (71) patients with confirmed S. aureus bacteremia/endocarditis received a single two hour infusion of CF-301 in addition to background SOC antibacterial therapy (CF-301 treatment group), e.g. intravenous vancomycin or daptomycin (6 mg per kg intravenously once per day for six weeks) and 45 patients with confirmed S. aureus bacteremia/endocarditis received standard of care antibiotics alone (placebo group). These 116 patients constituted the microbiological intent to treat (mITT) population of a Phase II clinical study and was the primary efficacy analysis population. The primary efficacy endpoint was the clinical responder rate (CRR) at Day 14. Diagnosis and clinical outcomes were determined by a blinded Adjudication Committee.

Results

The average patient was white, male and approximately 56 years of age (67.8%). A total of 38.8% of CF-301-treated and 35.5% of placebo patients, respectively, had a MRSA infection. The majority of patients in both treatment groups had bacteremia (77.5% of the treatment group and 86.7% of the placebo group); however, there was an unequal distribution of patients with left-sided endocarditis between the treatment groups. A total 15.5% of CF-301-treated patients had left-sided endocarditis compared to 6.7% of placebo patients. The CRR was 70.4% for the CF-301 treatment group and 60% for the placebo group (p=0.314).

In a prespecified analysis among MRSA-infected patients, the CRR in the group treated with CF-301 and standard of care antibiotics was about 40% higher than the CRR in patients treated with standard of care antibiotics alone (74.10% vs 31.3%; p=0.01). CRRs in the subset with bacteremia/right-sided endocarditis were 80% and 59.5%, for the CF-301 treatment group and placebo group, respectively (p=0.0²⁸). In patients with bacteremia alone, CRRs were 81.8% and 61.5% for the CF-301 treatment group and the placebo group, respectively (p=0.035). Among patients who received CF-301, the incidence of treatment emergent adverse events (TEAEs), was balanced between the groups (88.9% of the treatment group and 85.1% of the placebo group) as were serious TEAEs (47.2% of the treatment group and 51.1% of placebo group). 19.4% of the treatment group and 14.9% of the placebo groups died during the period from study drug administration through 28 days after the end of standard of care antibiotic treatment. There were no reports of hypersensitivity to CF-301 and no patients discontinued a study drug in either treatment group.

The results from this example demonstrate that the addition of a single dose of CF-301 during standard of care antibiotic treatment provides clinically meaningful improvements in responder rates compared to antibiotics alone for the treatment of MRSA bacteremia including endocarditis. Further, the addition of CF-301 to a standard of care antibiotic regimen was well-tolerated.

The present disclosure is not to be limited in scope by the specific embodiments described herein. Indeed, various modifications of the methods and components used therein in addition to those described herein will become apparent to those skilled in the art from the foregoing description.

All patents, applications, publications, test methods, literature, and other materials cited herein are hereby incorporated by reference.

### Example 4. Impact of Dose-Administration of CF-301 in Addition to DAP in an Experimental Infective Endocarditis (IE) Model Due to MRSA

Materials and Methods

A model of left-sided catheter-induced IE due to MRSA in rabbits (Li et al. The Journal of infectious diseases 2018, 218, 1367-1377) was used to examine the efficacy of CF-301 and DAP alone, and CF-301 in combination with DAP. The MRSA strain used in this example was MW2 (CA-MRSA; USA400; MIC (μg/ml)—DAP (0.5) CF-301 (1.0), see Indiani et al. Antimicrob. Agents Chemother. 2019, 63 doi:10.1128/AAC.02291-18 and Schuch et al. The Journal of infectious diseases 2014, 209, 1469-1478).

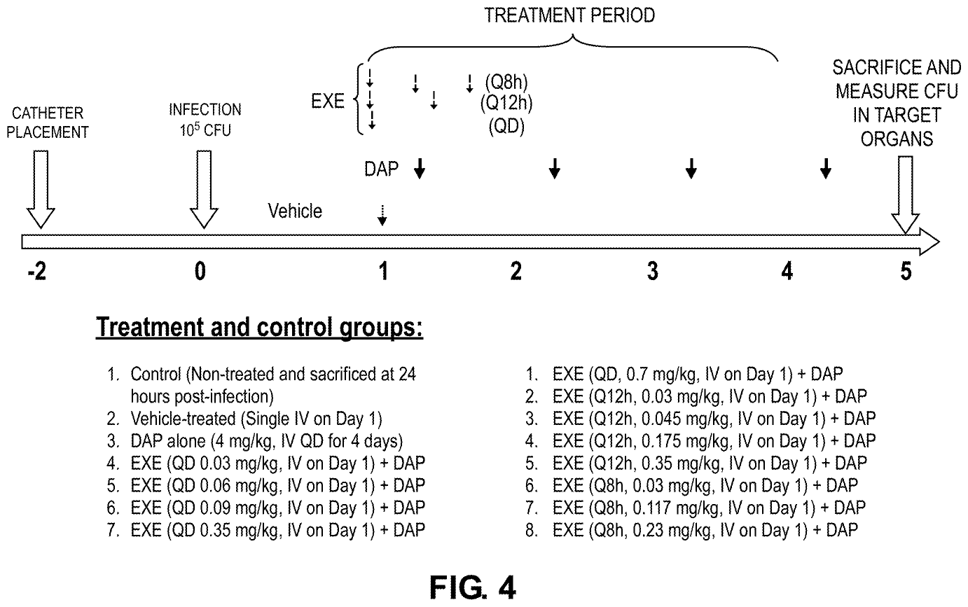

Briefly, female New Zealand White rabbits (Harlan Laboratories; 2.3 to 2.5 kg body weight) underwent transcarotid-transaortic valve catheterization, and IE was induced by IV infection of ˜1-2×10⁵ cfu of MW2 at 48 hours (h) after catheterization. At 24 h post-infection, animals were randomized into one of 15 groups: 1) controls; 2) vehicle controls given once daily (QD); 3-15) DAP alone (at 4 mg/kg iv QD×4 day; this dose yields significant but modest clearance of MRSA in experimental IE); DAP+CF-301 (given as an IV dose on the first day of DAP treatment only by 5-10 minutes slow bolus at (mg/kg): 0.70 QD, 0.35 Q12, 0.23 Q8 h, 0.35 QD, 0.175 Q12 h, 0.117 Q8 h, 0.09 QD, 0.045 Q12 h, 0.03 Q8 h, 0.06 QD, 0.03 Ql2 h or 0.03 QD. See also FIG. 4.

On day 5, animals were sacrificed, and target tissues (cardiac vegetations, kidney and spleen) were removed and quantitatively cultured. Tissue MRSA counts are given as the mean log₁₀ CFU/g of tissue±SD).

A two-tailed Student's t test was used to analyze the tissue MRSA counts between different groups. P values <0.05 were considered significant. No adjustment was made for all the P values reported in this study.

Results

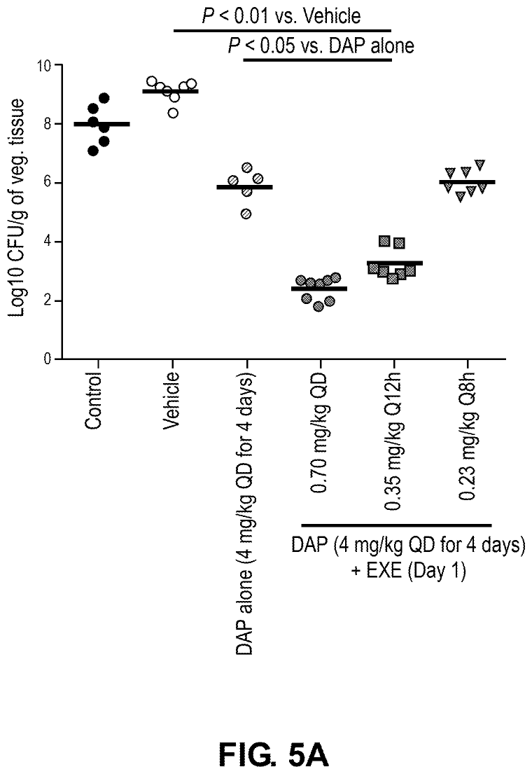

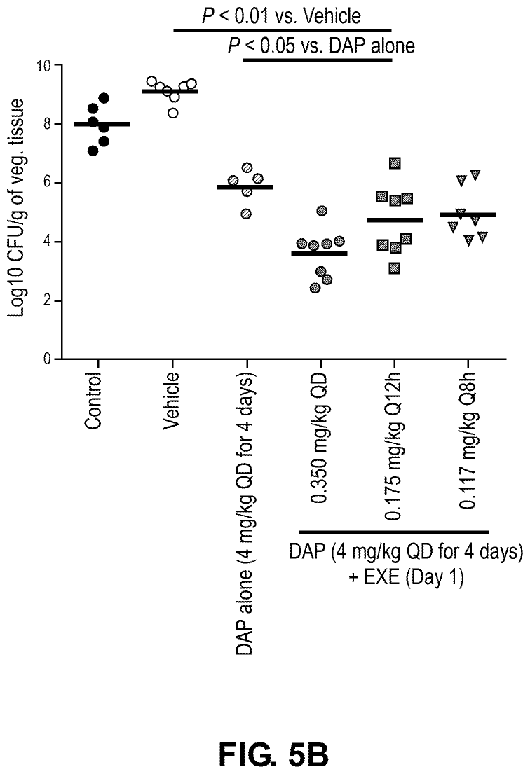

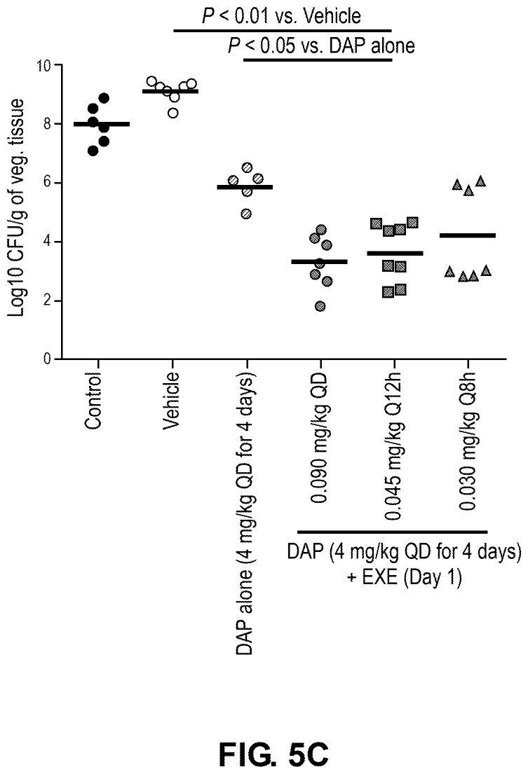

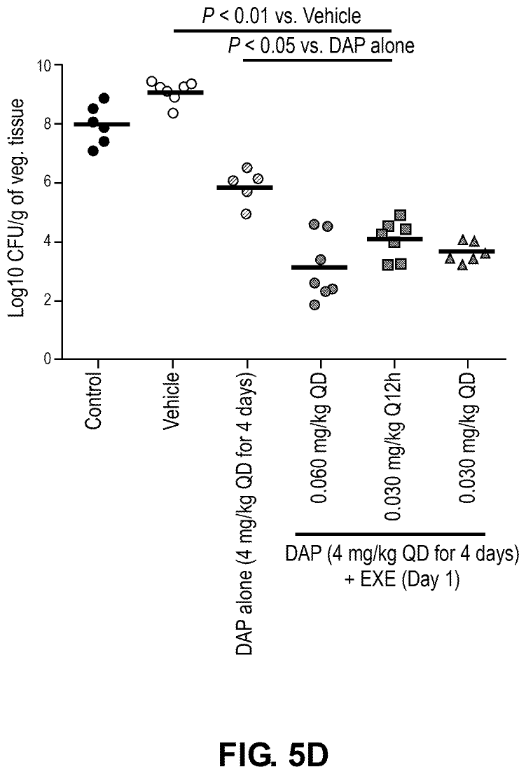

Treatment with DAP alone caused about 2-3 log₁₀ cfu/g reduction in MRSA densities in all three target tissues vs vehicle controls. All CF-301 doses given in addition to DAP, even at the lowest CF-301 dose (0.03 mg/kg), significantly reduced MRSA densities further in all target tissues vs DAP alone (about 3 log₁₀ cfu/g) and vehicle control groups (about 6 log₁₀ cfu/g). FIGS. 5A-5D and Table 5. In general, DAP plus CF-301 given as a single dose (“SD”), surprisingly, trended towards better microbiologic efficacy than CF-301 given at Q12 h or Q8 h, although this difference was not statistically significant.

These results demonstrate that CF-301, given at multiple dose strategies and at different dose-regimens, in addition to sublethal DAP, had significant efficacy in further decreasing MRSA densities in relevant target tissues in the IE model (vs DAP-alone and untreated controls). DAP plus a single dose of CF-301 trended to better efficacy than when it was administered in fractionated dose-strategies.

TABLE 5
Mean (±SD) MRSA Densities in Other Tissues
(Kidneys and Spleen) in the Rabbit IE Model.

| | Dose Level | Dose | Mean Log₁₀ CFU/g tissue ± SD | |
|---|---|---|---|---|
| Treatment | (mg/kg | frequency | Kidneys | Spleen |
| Control | — | — | 7.23 ± 0.93 | 6.98 ± 0.56 |
| Vehicle | 0 | QD | 8.23 ± 0.58 | 7.90 ± 0.48 |
| DAP | 4 | QD | 4.62 ± 1.16^{a} | 4.04 ± 0.87^{a} |
| CF-301/DAP | 0.7/4 | SD/QD | 2.02 ± 0.45^{a} | 2.14 ± 0.51^{a} |
| CF-301/DAP | 0.35/4 | Q12 h/QD | 2.41 ± 0.45^{a} | 2.37 ± 0.64^{a} |
| CF-301/DAP | 0.23/4 | Q8 h/QD | 5.03 ± 0.14^{a} | 4.95 ± 0.18^{a} |
| CF-301/DAP | 0.35/4 | SD/QD | 2.87 ± 0.42^{a} | 2.82 ± 0.58^{a} |
| CF-301/DAP | 0.175/4 | Q12 h/QD | 3.85 ± 0.51^{a} | 3.90 ± 0.33^{a} |
| CF-301/DAP | 0.117/4 | Q8 h/QD | 4.60 ± 0.50^{a} | 3.93 ± 0.31^{a} |
| CF-301/DAP | 0.09/4 | SD/QD | 3.45 ± 0.55^{a} | 3.20 ± 0.74^{a} |
| CF-301/DAP | 0.045/4 | Q12 h/QD | 3.06 ± 0.20^{a} | 2.99 ± 0.55^{a} |
| CF-301/DAP | 0.03/4 | Q8 h/QD | 3.34 ± 0.94^{a} | 3.52 ± 0.80^{a} |
| CF-301/DAP | 0.06/4 | SD/QD | 2.82 ± 0.40^{a} | 3.05 ± 0.42^{a} |
| CF-301/DAP | 0.03/4 | Q12 h/QD | 3.73 ± 0.33^{a} | 3.59 ± 0.38^{a} |
| CF-301/DAP | 0.03/4 | SD/QD | 3.05 ± 0.22^{a} | 3.10 ± 0.43^{a} |

Values in bold font indicate P < 0.05 vs. DAP alone;
^{a}P < 0.01 vs. Vehicle

### Example 5—Target Attainment of CF-301 to Determine Optimal Doses for Adult Patients with Staphylococcus aureus (S. aureus) Bloodstream Infections (Bacteremia) Including Endocarditis

A population pharmacokinetic (PPK) model was developed with data from 72 human patients presenting with S. aureus bacteremia infections to determine target attainment (TA) simulations for optimal doses of CF-301. The patients were administered CF-301 along with standard-of-care antibiotics. CF-301 was administered as a single 2-hour infusion of 0.25 mg/kg or 0.12 mg/kg for patients with a creatine clearance of less than 60 mL/minute, including patients on dialysis. The PPK model was used for TA simulations of various intravenous infusion regimens, as described below.

A three-compartment model was determined to best fit the data, and parameters were well-estimated. Clearance was 4.2 Liters (L)/hour (hr) with a relative standard error (RSE) of 5.5%, and central compartment (V_{c}) was 4.5 L with an RSE of 8.2%. Total volume distribution was 20.2 liters. Values were lower than those estimated previously in healthy subjects, CL=7.1 L/hr and volume distribution (V_{d}) 27.7 L. Creatine clearance was a clinically meaningful covariate. Patients with moderate and severe renal impairment are expected to have 1.3 to 2-fold higher AUC₀₋₂₄ or Cₘₐₓ than patients with normal renal function. Age was statistically significant on peripheral clearance, but not clinically meaningful (less than 4% effect on AUC₀₋₂₄ or Cₘₐₓ).

TA simulations were stratified by renal function performed across a range of fixed and weight-based doses. In patients with normal renal function or mild impairment, doses of 18 mg 2 hour IV infusion result in Cₘₐₓ and AUC₀₋₂₄ of 1254 ng/ml and 3026 ng*hr/mL, respectively. End-stage renal disease (ESRD) patients, including hemodialysis, a dose of 8 mg 2-hr IV infusion result in Cₘₐₓ and AUC₀₋₂₄ of 910 ng/mL and 3109 ng*hr/mL, respectively. These exposures place >99% subjects above the expected efficacious thresholds of AUC/MIC>0.2 established in animals.

The PPK model described the PK of CF-301 in patients adequately. CL and V_{d} were estimated to be 40% and 17% lower, respectively, than those in healthy subjects. CrCl was determined to be the only clinically meaningful covariate requiring dose adjustment. TA assessments identified doses that achieve the minimum efficacy.

1. A method of treating or preventing infective endocarditis due to Gram-positive bacteria in a subject, which method comprises:
administering a therapeutically effective amount of one or more antibiotics and a PlySs2 lysin comprising the amino acid sequence of SEQ ID NO: 18, SEQ ID NO: 2 or a variant thereof having at least 80% identity to SEQ ID NO: 2 or SEQ ID NO: 18, wherein the variant comprises bactericidal and/or bacteriostatic activity against the Gram-positive bacteria, and wherein the one or more antibiotics and the PlySs2 lysin are administered simultaneously or sequentially to the subject in need thereof in any order.

2. (canceled)

3. (canceled)

4. The method of claim 1, wherein the PlySs2 lysin and/or variant thereof and/or the one or more antibiotics is/are administered at a dose below the minimal inhibitory concentration (MIC) dose.

5. (canceled)

6. The method of claim 1, wherein the one or more antibiotics comprises one or more of a beta-lactam, an aminoglycoside, a glycopeptide, an oxazolidinone, a lipopeptide and a sulfonamide.

7. (canceled)

8. (canceled)

9. The method of claim 1, wherein the variant PlySs2 lysin comprises the amino acid sequence of any one of SEQ ID NOs. 3-17.

10. The method of claim 1, wherein the variant PlySs2 lysin has at least 90% identity to the amino acid sequence of SEQ ID NO: 2 or SEQ ID NO: 18.

11. (canceled)

12. (canceled)

13. The method of claim 1, wherein the endocarditis is right-sided endocarditis.

14. The method of claim 1, wherein the endocarditis is prosthetic valve endocarditis.

15. (canceled)

16. (canceled)

17. The method of claim 1, wherein the subject is (i) an intravenous drug user, (ii) a subject with cardiac device infection, (iii) a subject using central venous catheters, (iv) a subject with Human Immunodeficiency Virus (HIV), and/or (v) a subject having congenital heart disease.

18. The method of claim 1, wherein the infective endocarditis comprises a biofilm.

19. The method of claim 1, wherein the one or more antibiotics comprises vancomycin, penicillin, daptomycin and/or linezolid.

20. (canceled)

21. (canceled)

22. (canceled)

23. (canceled)

24. The method of claim 1, wherein the Gram-positive bacteria comprise a Staphylococcus bacteria and/or a Streptococcus bacteria.

25. The method of claim 1, where the Gram-positive bacteria comprise coagulase-negative staphylococci (CoNS).

26. (canceled)

27. The method of claim 1, wherein the Gram-positive bacteria comprise Methicillin-Sensitive Staphylococcus aureus (MSSA) and/or Methicillin-Resistant Staphylococcus aureus (MRSA).

28. The method of claim 1, wherein the Gram-positive bacteria comprise Staphylococcus aureus.

29. (canceled)

30. (canceled)

31. The method of claim 1, wherein the Gram-positive bacteria comprise Staphylococcus haemolyticus, Staphylococcus lugdunensis, Staphylococcus capitis, Staphylococcus hominus, Staphylococcus warneri, Staphylococcus pseudintermedius, Staphylococcus sciuri, and Staphylococcus hyicus Streptococcus mitis, Streptococcus intermedius, and/or Streptococcus salivarius.

32. (canceled)

33. (canceled)

34. (canceled)

35. (canceled)

36. The method of claim 1, wherein the Gram-positive bacteria comprises an antibiotic resistant Gram-positive bacteria and/or an antibiotic sensitive Gram-positive bacteria.

37. (canceled)

38. The method of claim 1, wherein the PlySs2 lysin is administered to the subject as dose fractions of a single dose.

39. The method of claim 38, wherein each dose fraction is administered every eight hours for one day or every twelve hours for one day.

40. (canceled)

41. (canceled)

42. The method of claim 1, wherein the PlySs2 lysin or variant thereof is administered intravenously in a single dose.

43. The method of claim 42, wherein the dosage ranges from about 0.1 mg/kg to about 0.3 mg/kg.

44. The method of claim 1, wherein the variant PlySs2 lysin has at least 95% identity to the amino acid sequence of SEQ ID NO: 2 or SEQ ID NO: 18.

45. The method of claim 1, wherein the variant PlySs2 lysin is the amino acid sequence of SEQ ID NO: 18.

46. The method of claim 1, wherein the PlySs2 or variant thereof is formulated as a single bolus for injection.

47. The method of claim 1, wherein the infective endocarditis comprises recurrent infective endocarditis.

48. The method of claim 1, wherein the infective endocarditis is a cardiac device infection.

49. The method of claim 43, wherein the PlySs2 lysin is the amino acid sequence of SEQ ID NO: 18 and the dosage is selected from 0.25 mg/kg and 0.12 mg/kg.

50. The method of claim 43, wherein the PlySs2 lysin is the amino acid sequence of SEQ ID NO: 18 and the dosage is selected from 18 mg or 8 mg.

51. The method of claim 50, wherein administration of a single dose comprising 18 mg of the PlySs2 lysin of amino acid sequence of SEQ ID NO: 18 is by intravenous injection and provides a maximal plasma concentration of 3026 ng*hr/mL at about 0 to about 24 hours after administration and/or a mean Cmax of 1254 ng/hr of said PlySs2 lysin.

52. The method of claim 50, wherein administration of a single dose comprising 8 mg of the PlySs2 lysin of amino acid sequence of SEQ ID NO: 18 is by intravenous injection and provides a maximal plasma concentration of 3109 ng*hr/mL at about 0 to about 24 hours after administration and/or a mean Cmax of 910 ng/hr of said PlySs2 lysin.

53. A composition comprising a therapeutically effective amount of a PlySs2 lysin comprising the amino acid sequence of SEQ ID NO: 18, SEQ ID NO: 2 or a variant thereof having at least 80% identity to SEQ ID NO: 2,
wherein the variant comprises bactericidal and/or bacteriostatic activity against Gram-positive bacteria,
wherein the composition is formulated as a single dosage for intravenous injection, and wherein a dosage of the PlySs2 or variant thereof ranges from about 0.1 mg/kg to about 0.3 mg/kg.

54. The composition of claim 53, wherein the PlySs2 lysin is the amino acid sequence of SEQ ID NO: 18 and the dosage is 0.25 mg/kg.

55. The composition of claim 53, wherein the PlySs2 lysin is the amino acid sequence of SEQ ID NO: 18 and the dosage is 0.12 mg/kg.

56. The composition of claim 53, wherein administration of a single dose comprising 18 mg of the PlySs2 lysin of amino acid sequence of SEQ ID NO: 18 by intravenous injection provides a maximal plasma concentration of 3026 ng*hr/mL at about 0 to about 24 hours after administration and/or a mean Cmax of 1254 ng/hr of said PlySs2 lysin.

57. The composition of claim 53, wherein administration of a single dose comprising 8 mg of the PlySs2 lysin of amino acid sequence of SEQ ID NO: 18 by intravenous injection provides a maximal plasma concentration of 3109 ng*hr/mL at about 0 to about 24 hours after administration and/or a mean Cmax of 910 ng/hr of said PlySs2 lysin.
